(19) United States
(12) Patent Application Publication
(10) Pub. No.: US 2025/0302380 A1
(43) Pub. Date: Oct. 2, 2025

(54) Sleep Apnea Prediction Using Electrocardiograms and Machine Learning

(71) Applicant: iRhythm Technologies, Inc., San Francisco, CA (US)

(72) Inventors: Andrew David Gilbert, Salt Lake City, UT (US); Steven Patrick Szabados, Longmont, CO (US); Justin Thomas Cambra, Oakland, CA (US); Mohanram Ganesan, Middleton, WI (US); Rebecca Guinn Heinrich, Cary, NC (US)

(73) Assignee: iRhythm Technologies, Inc., San Francisco, CA (US)

(21) Appl. No.: 19/093,806

(22) Filed: Mar. 28, 2025

Related U.S. Application Data
(60) Provisional application No. 63/571,292, filed on Mar. 28, 2024.

(51) Int. Cl. A61B 5/00; A61B 5/145; A61B 5/256; A61B 5/364; G16H 10/60; G16H 50/20; G16H 50/30; G16H 50/70

(52) U.S. Cl. CPC A61B 5/4818; A61B 5/14542; A61B 5/256; A61B 5/364; A61B 5/6801; A61B 5/742; G16H 10/60; G16H 50/20; G16H 50/30; G16H 50/70; A61B 2560/0462; A61B 2562/0219

(57) ABSTRACT

Sleep apnea prediction using electrocardiograms and machine learning is described. In one or more implementations, a wearable monitoring device produces electrical potential measurements of a heart of a user during an observation period spanning multiple days. A sleep apnea classification of the user is predicted by providing the electrical potential measurements to one or more machine learning models as input. The one or more machine learning models are trained based on historical electrical potential measurements and historical outcome data of a user population to correlate patterns in electrical potential measurements to sleep apnea classifications. The sleep apnea classification may then be output, such as in a health report, via a user interface, as notification on a computing device, and so forth.

### RELATED APPLICATIONS

This application claims priority to U.S. Provisional Patent Application No. 63/571,292, filed Mar. 28, 2024, and titled “Sleep Apnea Prediction Using Electrocardiograms and Machine Learning,” the disclosure of which is hereby incorporated by reference in its entirety.

### BACKGROUND

Sleep apnea is a common sleep disorder characterized by repeated interruptions in breathing during sleep. These interruptions, called apneas (e.g., complete cessation of breathing) or hypopneas (e.g., partial cessation of breathing), can last from a few seconds to minutes and may occur thirty or more times per hour. There are a variety of types of sleep apnea such as obstructive sleep apnea (OSA) and central sleep apnea (CSA). OSA occurs when throat muscles of an individual relax which causes physical obstruction of the airway, while CSA occurs when the brain of the individual does not send proper signals to muscles that control breathing. A severity of sleep apnea is typically evaluated using an apnea-hypopnea index (AHI), which represents a number of apnea and/or hypopnea events per hour of sleep. A relatively higher AHI, for instance, corresponds to a relatively more severe sleep apnea.

Diagnosing sleep apnea can be challenging due to its manifestation during sleep when individuals are unaware of breathing anomalies. Further, symptoms of sleep apnea such as daytime fatigue, snoring, interrupted sleep, and so forth may be overlooked or incorrectly attributed to other factors, leading to underdiagnosis. A subjective nature of these symptoms can also mimic other conditions, which complicates an apnea identification process in the absence of specialized diagnostic tools. Conventional techniques for diagnosis include polysomnography (PSG), which requires an overnight stay in a sleep lab to monitor various physiological functions and thus poses logistical and financial barriers. In an effort to increase accessibility to diagnostic modalities, conventional home sleep tests have been developed, however these tests often fail to capture an inherent complexity of the condition, such as to ascertain a severity of sleep apnea, differentiate between the different types of sleep apnea (e.g., to differentiate between OSA and CSA), or generate meaningful insights based on collected data which limits the utility of these conventional techniques and further complicates the diagnosis process.

### BRIEF DESCRIPTION OF THE DRAWINGS

FIG. 1 is a block diagram of a non-limiting example of an environment that is operable to employ sleep apnea prediction using electrocardiograms and machine learning as described herein.

FIG. 2 depicts a non-limiting example of a wearable electrocardiogram monitoring device.

FIG. 3 depicts a non-limiting system in an example implementation of sleep apnea prediction using electrocardiograms and machine learning showing operation of the prediction system of FIG. 1 in more detail.

FIG. 4 depicts a non-limiting example of sleep apnea prediction using electrocardiograms and machine learning in which electric potential measurements are processed by a trained machine learning model to generate predictions.

FIG. 5 depicts a non-limiting example of sleep apnea prediction using electrocardiograms and machine learning in which sequences of intermediate outputs of the machine learning model are concatenated to generate a model output.

FIG. 6 depicts a system in a non-limiting example of sleep apnea prediction using electrocardiograms and machine learning in which the system generates a comprehensive health report for output that includes the sleep apnea classification.

FIG. 7 depicts a non-limiting example of sleep apnea prediction using electrocardiograms and machine learning in which a report that includes the sleep apnea classification is output in a user interface.

FIG. 8 depicts a non-limiting example of sleep apnea prediction using electrocardiograms and machine learning in which a report that includes the sleep apnea classification is output in a user interface.

FIG. 9 depicts a flow diagram depicting an algorithm as a step-by-step procedure in an example implementation that is performable by a processing device to generate a sleep apnea classification based on electrical potential measurements.

### DETAILED DESCRIPTION

Sleep apnea prediction using electrocardiograms and machine learning is described. The described systems, methods, and devices, for instance, detect and classify sleep apnea using wearable technology. In an example, a wearable monitoring device is configured to capture electrical potential measurements of a heart of a user, e.g., electrocardiogram (ECG) data, over an extended observation period, e.g., multiple days. These measurements may then be analyzed using one or more sleep apnea models or algorithms to predict a sleep apnea classification for the user. In one or more implementations, the one or more sleep apnea models or algorithms include a machine learning model that is trained for a sleep apnea classification task using historical electrical potential measurements and historical outcome data from a user population as training data. The trained machine learning model thus provides a data-driven approach to sleep apnea detection and classification capable of generation of a variety of insights not possible using conventional approaches.

By way of example, the wearable monitoring device is implemented as a patch that is configured to be worn by the user (e.g., on the user's chest or back) and includes one or more sensors that contact skin of the user to capture a variety of physiological measurements and/or bio-signals that include electrical potential measurements associated with heart activity of the user. These measurements may be time-sequenced and collected continuously at predetermined intervals during the observation period, e.g., per second, per minute, etc., which provides a detailed and comprehensive dataset for analysis by the machine learning model as further described below.

Further, the one or more sensors may be arranged in a specific configuration on the wearable monitoring device to optimize collection of the physiological measurements including the electrical potential measurements. For instance, the sensors of the wearable monitoring device are designed to maintain consistent contact with the skin throughout the observation period in a variety of conditions, such as during periods of physical activity or sleep for multiple days. This consistent skin contact ensures that the electrical potential measurements captured by the sensors are reliable and accurate, thereby improving accuracy of the sleep apnea classification. In some cases, the wearable monitoring device includes features to secure the device to the body and maintain a position of the sensors against skin of the user such as one or more adhesive materials, straps, and/or other suitable attachment means.

In some examples, the wearable monitoring device may include additional sensors to collect additional physiological measurements, such as but not limited to sensors to collect accelerometer data and/or oxygen saturation (SpO2) data. For example, accelerometer data is collected by the wearable monitoring device and is processed to determine sleep patterns of the user. For instance, the accelerometer data can be analyzed to identify periods of physical inactivity and/or reduced movement, which are indicative of sleep.

This information is leveraged to establish when the user is likely sleeping during the observation period. By identifying these sleep periods, the system can effectively filter the collected data, focusing analysis on relevant time frames when sleep apnea events are likely to occur, e.g., when a user is determined to be sleeping. This targeted analysis enhances accuracy of sleep apnea classification, such as to reduce a potential for false positives that might arise from analyzing data collected during periods of wakefulness or physical activity, as well as conserves computational resources that would otherwise be expended to analyze nonrelevant data.

In an additional or alternative example, SpO2 data is collected using a pulse oximeter included in the wearable monitoring device to measure a level of oxygen in the blood. This data is usable to validate insights derived from processing of the ECG data as changes in oxygen saturation levels can be indicative of apnea events. In some embodiments, the SpO2 data is further useful in detection and assessment of a severity of sleep apnea events, as blood oxygen levels often decrease during apnea events. By incorporating these additional physiological measurements, the techniques described herein support accurate detection and classification of sleep apnea and further provide a comprehensive understanding of the sleep quality and overall health status of a user.

Based on one or more of the various collected physiological measurements, a prediction system is configured to process the data collected by the wearable device to generate one or more sleep apnea classifications. The sleep apnea classifications can include a variety of insights, indications, and/or predictions, such as whether the user has sleep apnea or does not have sleep apnea, a type of sleep apnea detected (e.g., OSA and/or CSA), a severity of sleep apnea (e.g., no sleep apnea, mild sleep apnea, moderate sleep apnea, severe sleep apnea, etc.), a sleep apnea score (e.g., an apnea-hypopnea index (AHI) score) or score range, is at risk for developing sleep apnea, whether the user is predicted to experience adverse effects associated with sleep apnea (e.g., daytime fatigue, snoring, low blood oxygen, atrial fibrillation (AFib), cardiac arrhythmias, etc.), a confidence or confidence interval in the sleep apnea classification (e.g., a confidence in an AHI score output, a confidence in a predicted severity of sleep apnea output, etc.), an efficacy of apnea treatments that have been implemented, and so forth. In various examples, the sleep apnea classification further indicates a correlation between sleep apnea and additional physiological events, such as cardiac events.

In various embodiments, the prediction system leverages the machine learning model to generate the sleep apnea classification. The machine learning model, for instance, is trained for a sleep apnea classification task using historical electrical potential measurements (e.g., historical electrocardiograms) and historical outcome data of a user population (e.g., clinical diagnosis data for users associated with the historical electric potential measurements) as training data. Once trained, the model is configured to correlate patterns in electrical potential measurements to various sleep apnea classifications. A variety of model types, architectures, training schema and so forth are considered as further described in more detail below.

Once generated, the prediction system outputs the sleep apnea classification, such as in a report, via a user interface, as a notification on a computing device, and so forth. The sleep apnea classification, for instance, provides an indication of a user state during the observation period, such as whether the user experienced sleep apnea or not, a severity of sleep apnea, a type of sleep apnea (e.g., OSA or CSA) experienced, and so forth. The sleep apnea classification may be determined for the observation period as a whole, as well as for discrete intervals of time that the user sleeps during the observation period, e.g., every day, every hour, etc., providing a detailed analysis of the user's sleep patterns and potential sleep apnea events. The prediction system is further operable to output the sleep apnea classification upon completion of the observation period and/or in real time during the observation period.

Accordingly, the techniques described herein provide a variety of advantages and support functionality not possible using conventional techniques. For instance, by analyzing bio-signal data from a wearable device, these techniques support accessible and accurate identification of signs of sleep apnea at an early stage, such as before a user experiences noticeable symptoms. This early detection can lead to earlier intervention to prevent progression of the condition and mitigate adverse health effects.

Moreover, the described techniques provide a non-invasive approach to monitoring sleep patterns and potential sleep apnea episodes. Unlike traditional sleep studies, which often require an overnight stay in a sleep lab, the techniques described herein utilize a wearable monitoring device that can be worn by the user outside of a clinical setting, e.g., at home. The wearable monitoring device can monitor heart activity of the user over extended periods of time and provide a comprehensive view of sleep patterns and potential sleep apnea episodes.

Additionally, the use of machine learning modalities enables accurate and personalized sleep apnea classification and generation of insights based on a variety of physiological measurements that are not possible using conventional techniques. By analyzing individual-specific heart activity (as well as additional individual-specific physiological data), the machine learning models described herein can provide a personalized sleep apnea classification that is tailored to a health profile of a particular individual. This personalized approach can improve accuracy of the sleep apnea classification, which leads to effective treatment strategies. In this way, the techniques described herein support accurate detection and classification of sleep apnea which enables early clinical intervention, effective treatment, and improved health outcomes for users.

In some aspects, the techniques described herein relate to a method implemented by a processing device including: obtaining electrical potential measurements of a heart of a user generated by a wearable monitoring device during an observation period; generating a sleep apnea classification of the user by processing the electrical potential measurements using a machine learning model trained to correlate patterns in electrical potential measurements to sleep apnea classifications; and outputting the sleep apnea classification.

In some aspects, the techniques described herein relate to a method, wherein the sleep apnea classification includes an indication describing a state of the user during the observation period as having sleep apnea or not having sleep apnea.

In some aspects, the techniques described herein relate to a method, wherein the machine learning model is configured to determine a severity of sleep apnea based on the electrical potential measurements, and the sleep apnea classification includes an indication describing a state of the user during the observation period as having no sleep apnea, mild sleep apnea, moderate sleep apnea, or severe sleep apnea.

In some aspects, the techniques described herein relate to a method, wherein the machine learning model is configured to generate a sleep apnea score based on the electrical potential measurements, and the sleep apnea classification includes an apnea-hypopnea index (AHI) score that quantifies a number of apnea events and hypopnea events per hour of sleep of the user during the observation period.

In some aspects, the techniques described herein relate to a method, wherein the machine learning model is configured to determine a type of sleep apnea based on the electrical potential measurements, and the sleep apnea classification includes an indication describing a state of the user during the observation period as having obstructive sleep apnea (OSA) or central sleep apnea (CSA).

In some aspects, the techniques described herein relate to a method, wherein the generating the sleep apnea classification includes extracting one or more electrocardiogram (ECG) features based on the electrical potential measurements and providing the one or more electrocardiogram features to the machine learning model as input.

In some aspects, the techniques described herein relate to a method, further including obtaining one or more additional physiological measurements from the wearable monitoring device and wherein the generating the sleep apnea classification includes inputting the one or more additional physiological measurements to the machine learning model as input.

In some aspects, the techniques described herein relate to a method, wherein the one or more additional physiological measurements include accelerometer data or oxygen saturation measurements.

In some aspects, the techniques described herein relate to a method, further including training the machine learning model to perform a sleep apnea classification task using historical electrical potential measurements and historical outcome data of a user population as training data.

In some aspects, the techniques described herein relate to a processing device including: one or more processors; and memory having stored computer-readable instructions that are executable by the one or more processors to perform operations including: obtaining electrical potential measurements of a heart of a user collected by a wearable monitoring device during an observation period; generating a sleep apnea classification of the user by providing the electrical potential measurements to a machine learning model as input, the machine learning model trained using historical electrical potential measurements and historical outcome data of a user population to perform a sleep apnea classification task; and outputting the sleep apnea classification in a user interface of the processing device.

In some aspects, the techniques described herein relate to a processing device, wherein the wearable monitoring device includes one or more sensors to collect accelerometer data and the generating the sleep apnea classification includes processing the accelerometer data by the machine learning model to determine a sequence of sleep of the user during the observation period.

In some aspects, the techniques described herein relate to a processing device, wherein the wearable monitoring device includes one or more sensors to collect oxygen saturation data and the generating the sleep apnea classification includes processing the oxygen saturation data by the machine learning model to validate the electrical potential measurements.

In some aspects, the techniques described herein relate to a processing device, wherein the sleep apnea classification is output during the observation period.

In some aspects, the techniques described herein relate to a processing device, wherein the sleep apnea classification is output following the observation period.

In some aspects, the techniques described herein relate to a processing device, wherein the sleep apnea classification includes an indication of a type and a severity of sleep apnea.

In some aspects, the techniques described herein relate to a processing device, the operations further including generating, by the machine learning model, an indication of one or more predicted future adverse effect of sleep apnea based on the electrical potential measurements.

In some aspects, the techniques described herein relate to a system including: a wearable monitoring device that is wearable by a user to detect one or more physiological measurements of the user during an observation period, the one or more physiological measurements including electrical potential measurements of a heart of the user; and a computing device configured to: receive the one or more physiological measurements from the wearable monitoring device; generate a sleep apnea classification of the user by processing the one or more physiological measurements by a machine learning model trained to correlate patterns in electrical potential measurements to sleep apnea classifications; and output the sleep apnea classification.

In some aspects, the techniques described herein relate to a system, wherein the physiological measurements further include accelerometer data collected during the observation period or oxygen saturation data collected during the observation period.

In some aspects, the techniques described herein relate to a system, wherein the sleep apnea classification includes details associated with an individual apnea event detected during the observation period.

In some aspects, the techniques described herein relate to a system, the computing device further configured to: detect one or more cardiac arrythmias during the observation period based on the one or more physiological measurements; generate, using the machine learning model, a correlation between the sleep apnea classification and the one or more cardiac arrythmias; and generate a visual indication for output by the computing device of the correlation.

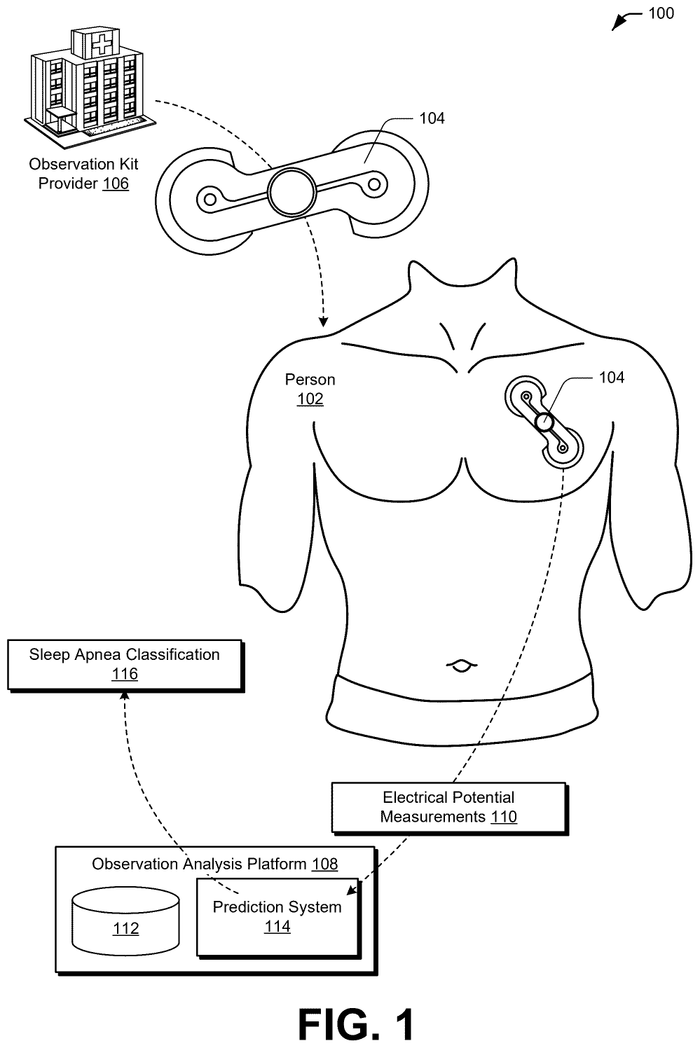

FIG. 1 is a block diagram of a non-limiting example 100 of an environment that is operable to employ sleep apnea prediction using electrocardiograms and machine learning as described herein. The illustrated example 100 includes person 102, who is depicted wearing a wearable monitoring device 104, e.g., a wearable electrocardiogram (ECG) monitoring device. The illustrated environment also includes an observation kit provider 106 and an observation analysis platform 108.

In the illustrated example 100, the wearable monitoring device 104 is depicted being provided by the observation kit provider 106 to the person 102, e.g., as part of an observation kit. The wearable monitoring device 104 may be provided as part of an observation kit, for instance, for the purpose of recording electrical activity of the heart of the person 102 over an observation period lasting multiple days. By way of example, the person 102 may have a magnitude of an electrical potential of the heart monitored over time to produce one or more electrocardiograms that are used to predict various sleep apnea classifications such as whether the person 102 has sleep apnea (e.g., obstructive sleep apnea (OSA) and/or central sleep apnea (CSA)), a severity of sleep apnea (e.g., normal to mild sleep apnea or moderate to severe sleep apnea), a sleep apnea score (e.g., an apnea-hypopnea index (AHI) score) or score range, is at risk for developing sleep apnea, whether the person 102 is predicted to experience adverse effects associated with sleep apnea (e.g., daytime fatigue, snoring, low blood oxygen, atrial fibrillation (AFib), or other arrhythmias, to name just a few), and/or a confidence or confidence interval in the sleep apnea classification (e.g., a confidence in an AHI score output, a confidence in a predicted severity of sleep apnea output, etc.).

Alternatively or additionally, the prediction system may output a time sequence indicating an observation or prediction of one or more apnea events, cardiac events and/or arrythmias, sleep disturbances, and/or characterizations of sleep disturbances over time. In some embodiments, the output may correspond to or include a prediction of a sequence of sleep, such as sleep versus awake, type of sleep or sleep stage (e.g., light sleep, deep sleep, REM sleep, etc.), a position of the person 102 during sleep, and so forth. It is to be appreciated that in variations, the output may correspond to or include one prediction (e.g., whether a person has sleep apnea), while in other variations the output may correspond to or include more than one prediction (e.g., whether a person has sleep apnea, type of sleep apnea, and confidence in one or both predictions). It is also to be appreciated that different combinations of multiple predictions may be output in variations.

In connection with the observation period, instructions may be provided to the person 102 that instruct the person 102 how to operate the wearable monitoring device 104 and/or how to behave (e.g., sleep) while wearing wearable monitoring device 104. In one or more implementations, the instructions may be provided as part of an observation kit, e.g., written instructions. Alternately or additionally, the observation analysis platform 108 may cause the instructions to be communicated to and output (e.g., for display and/or audio output) via a computing device associated with the person 102. The observation analysis platform 108 may provide these instructions for output after a predetermined amount of time of an observation period has lapsed (e.g., two days) and/or based on patterns in the electrical potential measurements obtained.

Although discussed as lasting multiple days, in one or more implementations, the observation period may be variable, such that when enough electrical potential measurements have been collected to accurately predict a sleep apnea classification for the person 102 the observation period may end. For example, in some cases the electrical potential measurements of the person 102 measured over a few hours may be processed to predict that the person 102 has sleep apnea with statistical certainty. In this case, the duration of the observation period may be a number of hours rather than multiple days. In alternative or additional examples, the observation period lasts multiple days to obtain data such that features can be extracted to describe day over day variations in electrical activity of the heart of the person 102 and to prevent erroneous predictions that account for or fail to account for anomalous measurements or observations.

To this end, the observation kit provider 106 may represent one or more of a variety of entities associated with obtaining a prediction regarding whether the person 102 has sleep apnea or is predicted to experience adverse effects of sleep apnea. For instance, the observation kit provider 106 may represent a provider of the wearable monitoring devices 104 and of a platform that monitors and analyzes sequences of electrical potential measurements (e.g., electrocardiograms) obtained therefrom, such as the observation analysis platform 108 when it also corresponds to the provider of the wearable monitoring device 104. Alternately or additionally, the observation kit provider 106 may correspond to a health care provider (e.g., a primary care physician, cardiologist, somnologist), a doctor office, a hospital, an insurance provider, a medical testing laboratory, or a telemedicine service, to name just a few. It is to be appreciated that these are just a few examples and the observation kit provider 106 may represent different entities without departing from the spirit or scope of the described techniques.

Given this, provision of the wearable monitoring device 104 to the person 102 may occur in various ways in accordance with the described techniques. For example, the wearable monitoring device 104 may be handed to the person 102, such as at a doctor office, hospital, medical testing laboratory, or a brick-and-mortar pharmacy, e.g., as part of an observation kit. Alternatively or additionally, the wearable monitoring device 104 may be applied to the person 102, such as to a chest or back region at a doctor office, hospital, medical testing laboratory, or brick-and-mortar pharmacy. Alternately or additionally, the wearable monitoring device 104 may be mailed to the person 102, e.g., from the provider of the wearable monitoring device 104, a pharmacy, a medical testing laboratory, a telemedicine service, and so forth. This is by way of example and not limitation, and the person 102 may obtain the wearable monitoring device 104 for an observation period in various ways.

Regardless of how the wearable monitoring device 104 is obtained by the person 102, the device is configured to monitor electrical activity of the heart of the person 102 over time, e.g., over the course of an observation period which lasts for a time period spanning multiple days. In at least one implementation, for instance, the wearable monitoring device 104 measures and records a magnitude of the electrical potential of the heart over the observation period. In this way, the magnitude and direction of electrical depolarization of the heart may be captured throughout the cardiac cycle. With the electrical activity measured and recorded by the wearable monitoring device 104, an electrocardiogram (ECG) can be produced, which is an electrogram of the heart plotting voltage versus time of the electrical activity of the heart.

The wearable monitoring device 104 may be configured in a variety of ways to monitor and record the electrical activity of the heart of the person. For instance, the wearable monitoring device 104 may be configured with one or more sensors, examples of which include one or more of a plurality of electrodes (e.g., that can be placed on the skin of the person), an accelerometer, a pulse oximeter (e.g., to measure and record oxygen saturation (SpO2) and/or produce a photoplethysmogram of the person 102), and so on. By way of example, a pair of electrodes of the wearable monitoring device 104 on the skin of the person 102 detect (e.g., continuously) electric potential difference between the two electrodes, enabling measurements of the heart's electrical potential to be measured and recorded, producing the electrical potential measurements 110.

As used herein, the term “continuous” used in connection with monitoring signals associated with sleep apnea (e.g., electrical activity of the heart of the person 102) may refer to an ability of a device to produce measurements substantially continuously, such that the device may be configured to produce the electrical potential measurements 110 at intervals of time (e.g., per hour, per 30 minute interval, per 5 minute interval, per 30 second interval, per second, per half second, and so forth), responsive to an event (e.g., an electrical signal reaching an inflection point such as a peak or a valley), and so forth. The functionality of the wearable monitoring device 104 to produce the electrical potential measurements 110 along with measurements and/or to record any of a variety of signals may vary without departing from the spirit or scope of the described techniques.

Although the wearable monitoring device 104 may be configured in a similar manner as wearable electrocardiogram monitoring devices used for monitoring and/or diagnosing cardiac activity (e.g., arrhythmias), in one or more implementations, the wearable monitoring device 104 may be configured differently than the devices used for monitoring and/or diagnosing cardiac activity. These different configurations may be deployed to control confounding factors of observation periods so that measurements are obtained that accurately reflect the effects of users' normal, day-to-day behavior how they sleep and thus the detection of events related to sleep apnea. This can include, for instance, limiting and/or preventing users from inspecting the measurements produced during the observation period. By preventing users from inspecting the electrical potential measurements 110 over the course of observation periods, the observation configurations further prevent users from seeing or otherwise observing sleep apnea-measurement events and changing behavior to counteract such events.

In one or more implementations, the wearable monitoring device 104 may be configured to offload measurements (e.g., electrical potential measurements and/or accelerometer data) during the course of the observation period. By way of example, the wearable monitoring device 104 may offload the measurements by transmitting them via a wired or wireless connection to an external computing device, e.g., at predetermined time intervals and/or responsive to establishing or reestablishing a connection with the computing device. In one or more implementations, the electrical potential measurements 110 and/or other data from the wearable monitoring device 104 may be compressed by the wearable monitoring device 104 for wireless transmission, e.g., using one or more of a variety of data compression techniques. Compression of the sensor data in this way can reduce battery usage of the wearable monitoring device 104 during the observation period and facilitate wear during assessments of sleep apnea.

To the extent that the wearable monitoring device 104 may be configured to store the electrical potential measurements 110 for an entirety of an observation period, in one or more implementations, the wearable monitoring device 104 may be configured without wireless transmission means, e.g., without an antennae to transmit the electrical potential measurements 110 wirelessly and without hardware or firmware to generate packets for such wireless transmission. Instead, the wearable monitoring device 104 may be configured with hardware to communicate the electrical potential measurements 110 via a physical, wired coupling. In such scenarios, the wearable monitoring device 104 may be “plugged in” to extract the electrical potential measurements 110 from the device's storage.

Accordingly, the wearable monitoring device 104 may be configured with one or more ports to enable wired transmission of the electrical potential measurements to an external computing device. Examples of such physical couplings may include micro universal serial bus (USB) connections, mini-USB connections, and USB-C connections, to name just a few. Although the wearable monitoring device 104 may be configured for extraction of the electrical potential measurements 110 via wired connections as discussed just above, in different scenarios, the wearable monitoring device 104 may alternately or additionally be configured to offload the electrical potential measurements 110 over one or more wireless connections.

Once the wearable monitoring device 104 produces the electrical potential measurements 110, the measurements are provided to the observation analysis platform 108. As noted above, the electrical potential measurements 110 may be communicated to the observation analysis platform 108 over wired and/or a wireless connection.

In scenarios where the observation analysis platform 108 is implemented partially or entirely on the wearable monitoring device 104, for instance, the electrical potential measurements 110 may be transferred over a bus from the device's local storage to a processing system of the device. In scenarios where the wearable monitoring device 104 is configured to generate a prediction of a sleep apnea classification by processing the electrical potential measurements 110, the wearable monitoring device 104 may also be configured to provide the predicted sleep apnea classification as output, e.g., by communicating the sleep apnea classification to an external computing device. In other scenarios, the electrical potential measurements 110 may be processed by an external computing device configured to predict sleep apnea classifications. For example, the electrical potential measurements 110 (and/or other measurements such as accelerometer data and oxygen saturation (SpO2) measurements) may be processed by a smartphone associated with the user, a smartphone or other dedicated device associated with the wearable monitoring device 104, and/or one or more server computers at a data center or other location that can be utilized by an entity associated with the wearable monitoring device 104, to name just a few.

In one or more implementations, the wearable monitoring device 104 is configured to transmit the electrical potential measurements 110 to an external device over a wired connection with the external device, e.g., via USB-C or some other physical, communicative coupling. Here, a connector may be plugged into the wearable monitoring device 104 or the wearable monitoring device 104 may be inserted into an apparatus having a receptacle that interfaces with corresponding contacts of the device. The electrical potential measurements 110 may then be obtained from storage of the wearable monitoring device 104 via this wired connection, e.g., transferred over the wired connection to the external device. Such a connection may be used in scenarios where the wearable monitoring device 104 is mailed by the person 102 after the observation period, such as to a health care provider, telemedicine service, provider of the wearable monitoring device 104, or medical testing laboratory. To this end, an observation kit (not shown) may include packaging (e.g., an envelope or box) to mail the wearable monitoring device 104 to such an entity after observation. Such a connection may also be used in scenarios where the wearable monitoring device 104 is dropped off by the person 102 after the observation period, such as at a doctor's office or hospital (or other establishment of a health care provider), a pharmacy, or a medical testing laboratory. Alternately or additionally, scenarios involving a wired connection may involve the person 102 plugging in the wearable monitoring device 104 to an external computing device after the testing period, e.g., using a cord provided as part of an observation kit. In these scenarios, the external computing device may communicate the electrical potential measurements 110 to the observation analysis platform 108 over a network (not shown), such as the Internet.

Alternately or additionally, provision of the electrical potential measurements 110 to the observation analysis platform 108 may involve the wearable monitoring device 104 communicating the electrical potential measurements 110 over one or more wireless connections. For example, the wearable monitoring device 104 may wirelessly communicate the electrical potential measurements 110 to external computing devices, such as a mobile phone, tablet device, laptop, smart watch, other wearable health tracker, and so on. Accordingly, the wearable monitoring device 104 may be configured to communicate with external devices using one or more wireless communication protocols or techniques.

By way of example, the wearable monitoring device 104 may communicate with external devices using one or more of Bluetooth (e.g., Bluetooth Low Energy links), near-field communication (NFC), Long Term Evolution (LTE) standards such as 5G, and so forth. Wearable monitoring devices 104 may be configured with corresponding antennae and other wireless transmission means in scenarios where the electrical potential measurements 110 are communicated to an external device for processing. In such scenarios, the electrical potential measurements 110 may be communicated to the observation analysis platform 108 in various manners, such as at predetermined time intervals (e.g., every day, every hour, or every five minutes), responsive to occurrence of some event (e.g., filling a storage buffer of the wearable monitoring device 104), or responsive to an end of an observation period, to name just a few.

Thus, regardless of where the observation analysis platform 108 is implemented (e.g., at the wearable monitoring device 104, at a smartphone associated with the person 102, or at a server device), the observation analysis platform 108 obtains the electrical potential measurements 110 produced by the wearable monitoring device 104. In one or more implementations, the observation analysis platform 108 also obtains other measurements produced by the wearable monitoring device 104 and/or any other devices used during the observation period, e.g., a smartwatch, chest strap, etc. As noted above, examples of such additional measurements include but are not limited to accelerometer data and/or oxygen saturation (SpO2) measurements.

In one or more implementations, the observation analysis platform 108 may be implemented in whole or in part at the wearable monitoring device 104. Alternately or additionally, the observation analysis platform 108 may be implemented in whole or in part using one or more computing devices external to the wearable monitoring device 104, such as one or more computing devices associated with the person 102 (e.g., a mobile phone, tablet device, laptop, desktop, or smart watch) or one or more computing devices associated with a service provider (e.g., a health care provider, a telemedicine service, a service corresponding to the provider of the wearable monitoring device 104, a medical testing laboratory service, and so forth). In the latter scenario, the observation analysis platform 108 may be implemented at least in part on one or more server devices.

In the illustrated example 100, the observation analysis platform includes a storage device 112. In accordance with the described techniques, the storage device 112 is configured to maintain the electrical potential measurements 110 and/or other measurements processed by the machine learning models in connection with predicting classifications of sleep apnea. The storage device 112 may represent one or more databases and also other types of storage capable of storing the electrical potential measurements 110 and/or other types of measurements. The storage device 112 may also store a variety of other data, such as demographic information describing the person 102, information about a health care provider, information about an insurance provider, payment information, prescription information, determined health indicators, account information (e.g., username and password), and so forth. The storage device 112 may also maintain data of other users of a user population.

In the illustrated example 100, the observation analysis platform 108 also includes prediction system 114. The prediction system 114 represents functionality to process the electrical potential measurements 110 to generate sleep apnea predictions, such as to predict whether the person 102 has sleep apnea or not, a type of sleep apnea (e.g., obstructive sleep apnea (OSA) and/or central sleep apnea (CSA)), a severity of sleep apnea (e.g., normal to mild sleep apnea or moderate to severe sleep apnea), a sleep apnea score (e.g., an apnea-hypopnea index (AHI) score) or score range, is at risk for developing sleep apnea, whether the user is predicted to experience adverse effects associated with sleep apnea (e.g., daytime fatigue, snoring, low blood oxygen, atrial fibrillation (AFib), or other arrhythmias, to name just a few), and/or a confidence or confidence interval in the sleep apnea classification (e.g., a confidence in an AHI score output, a confidence in a predicted severity of sleep apnea output, etc.). The prediction system 114 is further operable to generate a correlation between and/or identify co-occurrence of apnea events and cardiac events, e.g., one or more arrythmias.

Alternatively or in addition, the prediction system 114 may output one or more time sequences indicating an observation or prediction of one or more apnea events, sleep disturbances, and/or characterizations of sleep disturbances, over time. In various examples, the prediction system 114 may output a prediction of a sequence of sleep, such as sleep versus awake, type of sleep or sleep stage (e.g., light sleep, deep sleep, REM sleep, etc.). It is to be appreciated that in variations, the prediction system 114 may output one prediction (e.g., whether a person has sleep apnea), while in other variations the prediction system may output more than one prediction (e.g., whether a person has sleep apnea, type of sleep apnea, and confidence in one or both predictions). It is also to be appreciated that the prediction system 114 may output different combinations of multiple predictions in variations.

In accordance with the described techniques, the prediction system 114 is operable to utilize machine learning to predict sleep apnea classifications. For instance, any one or more of the above-noted predictions may be output by the prediction system 114. Use of machine learning may include, for instance, leveraging one or more models generated using machine learning techniques as well as using historical electrical potential measurements (e.g., historical electrocardiograms) and historical outcome data of a user population (e.g., whether the users are clinically diagnosed with sleep apnea).

By way of example and not limitation, the prediction system 114 may include one or more neural networks trained based on the historical electrical potential measurements and the historical outcome data of the user population. Examples of such neural networks include, but are not limited to, U-Net which is a convolutional neural network for biomedical image segmentation based on a fully convolutional neural network whose architecture is modified and extended to work with fewer training images and to yield more precise segmentation, and ResNet (a residual neural network) which is a deep learning model in which the weight layers learn residual functions with reference to the layer inputs and behave like a highway network whose gates are opened through strongly positive bias weights, to name a few. The prediction system 114 may include one or multiple machine learning models (e.g., an ensemble of models). Alternatively or additionally, the prediction system 114 may include logic (a machine learning model and/or other types of logic) to pre-process the obtained measurements, such as to extract various cardiovascular and/or other features from the sequences of measurements.

In one or more implementations, for example, the prediction system 114 augments the sleep apnea classification 116 with one or more ECG rhythm classifications, such as ECG rhythm classifications output from an ECG rhythm classification algorithm (e.g., classifier) which also receives the electrical potential measurements 110 (ECGs) as input. In one or more implementations, for instance, such an ECG rhythm classification algorithm is capable of prediction of an occurrence of cardiac events such as atrial fibrillation (AFib) events from the electrical potential measurements 110. In such implementations, the prediction system 114 may provide indications of predicted AFib events output by the ECG rhythm classification algorithm as an input to the one or more machine learning models of the prediction system 114 used to predict the sleep apnea classification 116.

Alternatively or additionally, the prediction system 114 may use indications of predicted AFib events output by the ECG rhythm classification algorithm to separate periods of time, e.g., of the electrical potential measurements 110, and use different classification algorithms for different periods, such as a sleep apnea classification algorithm (or model) trained for AFib during predicted time periods of AFib and a sleep apnea classification algorithm (or model) trained for no AFib during predicted time periods of non-AFib. Alternatively or additionally, the prediction system 114 may use one or more ECG rhythm classification algorithms to determine periods of time when the input signal (e.g., the electrical potential measurements 110) is of high enough quality to produce accurate sleep apnea classifications and when it is not high enough to produce accurate classifications.

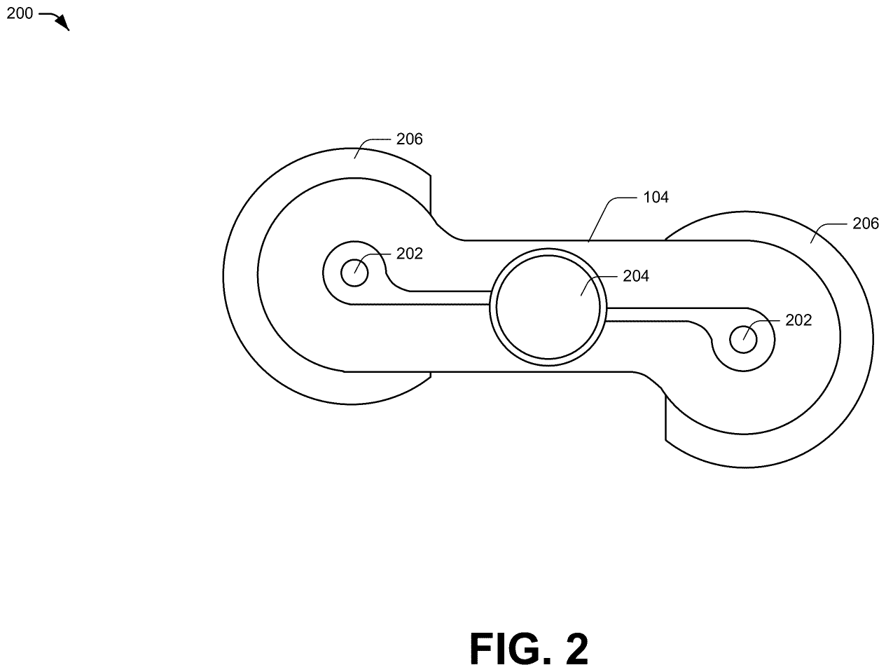

The illustrated example 100 also includes sleep apnea classification 116, which corresponds to the output of the prediction system 114. In accordance with the described techniques, the sleep apnea classification 116 may indicate whether it is predicted the person has sleep apnea, a type of sleep apnea, an AHI score, a confidence, a confidence interval, is predicted to experience adverse effects associated with sleep apnea, detailed information with respect to individual apnea events, and so on. The sleep apnea classification 116 may also be used to generate one or more notifications or user interfaces based on the classification, such as a report directed to a health care provider that includes the sleep apnea classification (e.g., that the person is predicted to have sleep apnea) or a notification directed to the person 102 that instructs the person to contact a health care provider. In the context of measuring electrical potential, e.g., continuously, and obtaining data describing such measurements, consider the following discussion of FIG. 2.

FIG. 2 depicts a non-limiting example 200 of a wearable electrocardiogram monitoring device. The illustrated example 200 depicts the wearable monitoring device 104.

In accordance with the described techniques, the wearable monitoring device 104 includes one or more sensors 202, examples of which include but are not limited to one or more pairs of electrodes, an accelerometer, a pulse oximeter, and sweat sensors, to name just a few. The wearable monitoring device 104 also includes a transmitter 204. In this example 200, the wearable monitoring device 104 further includes one or more adhesive portions 206. In operation, the wearable monitoring device 104 is configured to be applied to the skin via the one or more adhesive portions 206, such that the one or more sensors 202 are positioned to detect and record the electrical activity of the heart of the person 102, e.g., to produce an electrocardiogram (ECG and/or EKG). The wearable monitoring device 104 may be removed by peeling the one or more adhesive portions 206 off of the skin.

It is to be appreciated that the wearable monitoring device 104 and its various components are simply one form factor, and the wearable monitoring device 104 and its components may have different form factors without departing from the spirit or scope of the described techniques.

In one or more implementations, the wearable monitoring device 104 may include a processor and/or memory (not shown), the memory having stored computer-readable instructions that are executable by the processor to perform various operations. The wearable monitoring device 104, by leveraging the processor, may generate the electrical potential measurements 110 based on the communications with one or more sensors 202 that are indicative of the heart's electrical activity. In one or more implementations, the processor further generates one or more communicable packages of data that include one or more of the electrical potential measurements 110 and/or other measurements, such as accelerometer data and oxygen saturation (SpO2) measurements. Alternately or additionally, the processor produces and/or causes storage of other data, which may be used for predicting classifications of sleep apnea.

In implementations where the wearable monitoring device 104 is configured for wireless transmission, the transmitter 204 may transmit the measurements wirelessly as a stream of data to a computing device. In one or more implementations, for instance, the wearable monitoring device 104 is configured to transfer (e.g., transmit and/or receive) information (e.g., electrical potential measurements) via a Bluetooth Low Energy (BLE) connection. Alternately or additionally, the wearable monitoring device 104 may buffer the measurements (e.g., in memory) and cause the transmitter 204 to transmit the buffered measurements later at various intervals, e.g., time intervals (per second, per thirty second interval, per minute, per five minute interval, per hour, and so on), storage intervals (when the buffered measurements reach a threshold amount of data), and so forth.

Having discussed an example environment and an example wearable electrocardiogram monitoring device, consider the following example implementations of sleep apnea prediction using electrocardiograms and machine learning.

In one or more implementations, the classification of sleep apnea is performed post wear (e.g., after an observation period during which the wearable monitoring device 104 is worn). In at least one variation, the classification is performed for an overall and daily perspective if the person 102 is suspected of experiencing moderate/severe sleep apnea across the observation period (e.g., >3 days, up to 30 days) based on analysis of bio-signals from a wearable device that captures ECG, e.g., the wearable monitoring device 104.

In one or more implementations, an observation analysis platform 108 incorporates results of the analysis into a report (e.g., a PDF file) and/or presents such results via a digital portal or mobile application. In accordance with the described techniques, the results may be usable to identify those patients most appropriate to receive a follow-on sleep test. Further, in scenarios where the prediction system 114 is capable of accurately predicting the type of suspected sleep apnea (e.g., obstructive sleep apnea (OSA) and central sleep apnea (CSA)), this information can aid a physician in selecting a type of follow-on test to be performed (polysomnography (PSG) versus at home sleep test). Additionally, for patients where atrial fibrillation is also detected, the classification is intended to aid intervention decision making.

In one or more implementations, the classification of sleep apnea is performed during wear, e.g., while the wearable monitoring device 104 is worn by the person 102. In such implementations, the classification is performed regular or irregular intervals of time (e.g., daily and/or responsive to detection of events such as sleeping and/or resting) during wear from an overall and daily perspective if a patient is suspected of experiencing moderate/severe sleep apnea across the observation period (e.g., >3 days, up to 30 days) based on analysis of bio-signals from a wearable (e.g., the wearable monitoring device 104) that captures ECG data and is capable of uploading data during wear via wireless connection (BLE) to a mobile application.

In such implementations, analysis of the uploaded data is performed on the cloud and/or by a mobile application and results are then made presented via a digital portal or web application. To this end, the prediction system 114 (e.g., having the one or more machine learning models) may be implemented in variations at a computing device (e.g., smart phone) associated with the person 102 and/or by a server device in the cloud. The intent of providing sleep apnea insights while the wearable monitoring device 104 is worn is to shorten time to prediction of a classification of sleep apnea by allowing earlier in the patient care pathway prescription of a sleep test, e.g., a polysomnography (PSG) or additional at home sleep test. In the event the patient is utilizing an obstructive sleep apnea (OSA) intervention solution (i.e., a continuous positive airway pressure (CPAP) machine), the classifications output by the observation analysis platform 108 can also provide insight into compliance as well as effectiveness of the intervention.

In one or more implementations, the classifications produced by the observation analysis platform 108 (e.g., post wear) can indicate whether the person 102 is not predicted to experience sleep apnea over the observation period (e.g., >3 days, up to 30 days) based on analysis of bio-signals from a wearable patch that captures ECG, e.g., the wearable monitoring device 104. In one or more implementations, the observation analysis platform 108 incorporates results of the analysis into a report (e.g., a PDF file) and/or presents such results via a digital portal or mobile application, i.e., no sleep apnea. The intent of this case is to limit unnecessary expensive follow-on testing such as polysomnography (PSG). Additionally, for patients where atrial fibrillation is also detected, the classification is intended to aid intervention decision making.

In accordance with the described techniques, the prediction system 114 is or includes an artificial intelligence deep learned algorithm that is able to determine from electrocardiogram (ECG) at least one of if the person 102 experienced sleep apnea, a type of sleep apnea (e.g., obstructive sleep apnea (OSA) and/or central sleep apnea (CSA)), and/or an associated severity for each period (e.g., night) of sleep. In one or more implementations, the wearable monitoring device 104 and the observation analysis platform 108 are configured to enable the “streaming” of ECG (and/or other data) from the wearable monitoring device 104 to the observation analysis platform 108 for analysis. In at least one implementation, this involves leveraging either a mobile device associated with the person 102 or a device (e.g., a smartphone) provided in connection with the observation period that runs a mobile application. The mobile application may enable data to be uploaded from the wearable monitoring device 104 to the observation analysis platform 108, allowing physician review using a web-based portal. Using this technology along with the artificial intelligence sleep apnea classification algorithm, on a daily basis, determination of the presence and severity of sleep apnea can be provided during wear via the portal, a mobile application, and/or as an intermediate report.

In at least one example implementation, analysis of the data of the person 102 is performed after the observation period, e.g., after the wearable monitoring device 104 is worn by the person 102 and removed. In this scenario, a clinician may prescribe that the person 102 wear the wearable monitoring device 104. The wearable monitoring device 104 is applied to the person 102, such as for an observation period spanning multiple days. In one or more implementations, the wearable monitoring device 104 is applied for a period of time spanning from 3 days up to 30 days. During the observation period, the wearable monitoring device 104 monitors and records electrical activity of the heart of the person 102. In this scenario, the wearable monitoring device 104 stores measurements of the electrical activity (e.g., one or more electrocardiograms), such as in computer-readable storage of the wearable monitoring device 104. After the observation period, such as when the wearable monitoring device 104 is removed, the stored data (e.g., the measurements) is extracted from the wearable monitoring device 104. For example, the stored data is extracted from the wearable monitoring device 104 via streaming and/or direct download.

Bio-signal data from the wearable monitoring device 104 (e.g., electrical potential measurements, accelerometer data, and/or oxygen saturation (SpO2) measurements) is uploaded to an observation analysis platform 108, e.g., which in one or more implementations is implemented “in the cloud.” In accordance with the described techniques, the uploaded information is processed or otherwise analyzed for sleep apnea. For instance, the uploaded data is provided as input to the prediction system 114 (e.g., to artificial intelligence of the prediction system 114) to predict a classification of sleep apnea for the person (e.g., the presence or absence of sleep apnea, type of sleep apnea, adverse conditions experienced, severity of sleep apnea, and so on). In at least one implementation, the prediction system 114 is configured to process the uploaded data to identify and output for each period (e.g., night) of sleep a presence and/or severity of sleep apnea.

In one or more implementations, the observation analysis platform 108 compiles the results output by the prediction system 114 into a report. In at least one example, the report includes an overall indication of whether the person 102 experienced sleep apnea during wear of the wearable monitoring device 104, i.e., during the observation period. Additionally or alternatively, the report may include an indication of whether detected sleep apnea is determined to be normal to mild or moderate to severe. In one or more implementations, a determination of severity is based, at least in part, on an apnea-hypopnea index (AHI) generated based on the electrical potential measurements 110 obtained for the person 102 during the observation period.

Alternatively or additionally, the report is organized on a periodic basis, such as daily, nightly, or per interval of sleep. By way of example, in at least one implementation, the report includes indications of whether the person 102 experienced sleep apnea during each night's sleep while wearing the wearable monitoring device 104. Further, such a report may also include an indication of whether detected sleep apnea for each particular period is determined to be normal to mild or moderate to severe, such that different nights may be associated with different severities of sleep apnea. By way of example, the observation analysis platform 108 may associate a severity of normal to mild sleep apnea with a first night and may associate a severity of moderate to severe sleep apnea with a second night based on the electrical potential measurements 110 and/or features derived from the electrical potential measurements 110, e.g., AHI.

In various examples, the sleep apnea classification 116 may include detailed information about individual apnea events detected during the observation period. Accordingly, the report may include a “breakdown” of an identified apnea event that includes information such as a timestamp, duration, and severity of the event. For example, the report may indicate “apnea event detected at 2:15 AM on September 14, duration of 25 seconds, classified as severe obstructive sleep apnea.”

The report may also provide contextual information for each individual apnea event, such as a body position, heart rate, oxygen saturation levels, and/or additional physiological data detected before, during, and after the apnea event. In some implementations, the report may include visualizations of an ECG waveform or other physiological signals associated with each apnea event. The system is further configurable to categorize and group similar apnea events, such as to identify physiological or temporal patterns to generate clusters of apnea events based on various characteristics.

In one or more implementations, the observation analysis platform 108 for each night charts periods of predicted sleep apnea along with one or more of the following: arrhythmias experienced, heart rates, wake/sleep (rest periods). In this way, the observation analysis platform 108 can correlate (e.g., overlay) cardiac findings with sleep apnea events. In one or more implementations, the observation analysis platform 108 generates and outputs a sleep apnea and rhythm proportion analysis. In one example, this analysis is output as at least part of a report that contains a percentage of time sleep apnea occurs in rhythms observed, including but not limited to sinus and atrial fibrillation. Alternatively or additionally, the report indicates a predicted type of sleep apnea for detected events, e.g., obstructive sleep apnea (OSA) and/or central sleep apnea (CSA). This insight can be used to aid a clinician with a type of sleep test to prescribe for further analysis, e.g., polysomnography (PSG) or at-home test.

In at least one example implementation, analysis of the data for the person 102 is performed during the observation period, e.g., while the wearable monitoring device 104 is worn by the person 102. Like in the above scenario, a clinician may prescribe that the person 102 wear the wearable monitoring device 104, which can be applied to the person 102 for an observation period spanning multiple days.

In contrast to the scenario discussed above where analysis of the measurements is performed after the observation period, in one or more implementations, the wearable monitoring device 104 communicates measurements of the electrical activity (e.g., one or more electrocardiograms) and/or other data for analysis during the observation period. For example, the wearable monitoring device 104 communicates electrical potential measurements 110 to an external computing device for analysis. By way of example, the wearable monitoring device 104 uploads one or more electrocardiograms and/or other bio-signal data (e.g., accelerometer data, oxygen saturation (SpO2) measurements, etc.), such as to the cloud (e.g., the observation analysis platform 108) via a mobile application connected to the wearable monitoring device 104 over a Bluetooth Low Energy (BLE) connection during wear. In such implementations, the prediction system 114 analyzes the uploaded data during the observation period (e.g., from a previous night's sleep) to predict classifications of a presence or absence of sleep apnea, type of sleep apnea, and/or severity of sleep apnea.

In one or more implementations, the classification findings are captured in a daily report and/or they may be available via a user interface of a customer portal/mobile application. In one or more implementations, the information presented in a daily report may include whether sleep apnea was present, a severity of sleep apnea, when during sleep was sleep apnea observed, a heart rate and rhythms present during the period of sleep apnea, and so on.

Similar to the scenario where the uploaded data is analyzed after the observation period, in one or more scenarios where the data is uploaded and analyzed at least in part during the observation period, the observation analysis platform 108 for each night charts periods of predicted sleep apnea along with one or more of the following: arrhythmias experienced, heart rates, wake/sleep (rest periods). In this way, the observation analysis platform 108 can correlate (e.g., overlay) cardiac findings with sleep apnea events. In one or more implementations, the observation analysis platform 108 generates and outputs a sleep apnea and rhythm proportion analysis. In one example, for instance, this analysis is output as at least part of a report (e.g., daily report) that contains a percentage of time sleep apnea occurs in rhythms observed, including but not limited to sinus and atrial fibrillation. Alternatively or additionally, the report (e.g., the daily report) indicates a predicted type of sleep apnea for detected events, e.g., obstructive sleep apnea (OSA) and/or central sleep apnea (CSA). As noted above, this insight can be used to aid a clinician with a type of sleep test to prescribe for further analysis, e.g., polysomnography (PSG) or at-home test.

In one or more implementations, the prediction system 114 outputs a binary sleep apnea classification indicating whether the person 102 has moderate to severe sleep apnea or not. As mentioned above, in at least one variation, the output of the prediction system 114 is a daily indication of whether the person 102 experienced moderate to severe sleep apnea for the respective day, such that for a first day the output can indicate that a person did not experience sleep apnea and for a second day the output can indicate that the person did experience sleep apnea. In accordance with the described techniques, the output (e.g., the sleep apnea classification 116) informs healthcare providers (e.g., physicians) of patients with moderate to severe sleep apnea without having to perform expensive and inconvenient sleep tests, such as a polysomnography (PSG). Further, this enables a follow-on diagnostic test prescription to confirm a predicted classification and enables intervention.

In one or more implementations, the prediction system 114, implemented with the artificial intelligence algorithm and using the electrical potential measurements 110 from the wearable monitoring device 104, is configured to: predict classifications of moderate to severe sleep apnea (e.g., based on an AHI classification); generate a nightly (or other interval of time) AHI score; output a classification of mild, moderate, or severe AHI; report individual events and/or regions of high AHI in the electrical potential measurements 110 (e.g., one or more electrocardiograms) provided by the wearable monitoring device 104; distinguish between types of sleep apnea experienced (OSA vs. CSA); identify arousals or other sleep disordered breathing; identify sleep stages, and/or predict a measure of confidence (e.g., in AHI scores), to name a few. Regarding a measure of confidence or confidence interval, in one or more implementations, the prediction system 114 determines and outputs measures of confidence and/or confidence intervals derived and output by the one or more machine learning models. Alternatively or additionally, the prediction system 114 derives the measures of confidence and/or confidence intervals from other metrics, such as a quality score of one or more ECG signals or other derived cardiovascular features. By way of example, ECG signal quality may be derived from the ECG signals or measurements input into the prediction system 114 (e.g., electrical potential measurements 110) and/or of historical ECG signals or measurements captured from the user population.

Accordingly, use of the wearable monitoring device 104 and the prediction system 114 enable detection of various types and/or severities of sleep apnea, and thus support physician intervention to prescribe one or more apnea mitigations such as use of a CPAP machine.

Broadly, sleep apnea occurs when breathing repeatedly stops and starts during sleep. Each stop and start of breath may be considered an “event.” The term “hypopnea” refers to shallow breathing, and the term “apnea” refers to the cessation of breathing. Different causes of sleep apnea include “obstructive” sleep apnea where an airway is blocked, “central” sleep apnea where the brain stops sending proper signals to the muscles that control breathing, and mixed sleep apnea which is some combination of obstructive and central sleep apnea.

The apnea-hypopnea index (AHI) is defined as the number of apnea/hypopnea events occurring per hour of sleep. In various examples, “normal” sleep apnea is defined as AHI≤5, “mild” sleep apnea is defined as 5<AHI≤15, “moderate” sleep apnea is defined as 15<AHI≤30, and “severe” sleep apnea is defined as AHI>30. In various implementations, AHI does not distinguish between types of events, e.g., whether an apnea event is OSA or CSA. In one or more implementations, the apnea-hypopnea index is calculated in accordance with the following:

$$A H I = \frac{\sum O S A + \sum C S A + \sum M S A + \sum Hypopnea}{Total Sleep Time}$$

In one or more implementations, the prediction system 114 is trained with a dataset from a population of users. The data may include the electrical potential measurements (e.g., electrocardiograms) from the users of the population collected during one or more studies. Like the data input to the prediction system 114 during operation, the data set used to train the models or algorithms of the prediction system 114 from the users of the user population may be produced based on electrical activity detected by electrodes, e.g., single lead ECG.

Notably, AHI scores vary nightly between patients. For instance, there is significant inter-night variability in AHI scores, particularly among patients with severe sleep apnea. Thus, in some examples patients are monitored for multiple days to successfully capture the occurrence of sleep apnea events. The wearable monitoring device 104, which supports data collection during an extended period of time, thus enables patients to be monitored for several days.

In one or more implementations, an initial sleep apnea assessment made using the electrical potential measurements 110 from the wearable monitoring device 104 may be performed during the observation period to determine if a more accurate assessment with more sensors should be subsequently performed. Thus, such an initial assessment may involve wearing a simple ECG patch (e.g., the wearable monitoring device 104) for one or multiple nights of sleep to see if a sleep apnea classification of moderate to severe sleep apnea is output. If a sleep apnea classification of moderate to severe sleep apnea is output for the initial assessment, then additional sensors of the wearable monitoring device 104 may be activated or added for the assessment so that they can be used to produce additional data subsequently.

For example, after the initial assessment, an additional sensor of the wearable monitoring device 104, such as a pulse oximeter to produce oxygen saturation (SpO2) measurements, may be turned on or otherwise activated to begin producing respective measurements for a subsequent stage of the assessment. Such additional sensors may be enabled or otherwise turned on or off for subsequent stages of the assessment in order to produce predictions having a higher accuracy during such subsequent stages.

Moreover, enabling such sensors to be selectively turned on (or off) can extend a battery life of monitoring devices (e.g., the wearable monitoring device 104), since some sensors (e.g., pulse oximeters) use relatively more battery than other sensors. Thus, such sensors may be enabled when it is determined that use of such sensors will result in increased accuracy of the prediction(s). Alternatively or additionally, if the wearable monitoring device 104 is not configured with such additional sensors, where the additional sensors can be selectively turned on (e.g., activated) or turned off (e.g., deactivated), then the person 102 may obtain the additional sensors in other ways, e.g., the additional sensors may be delivered to the person 102 or the person 102 may pick up such additional sensors. Once delivered or otherwise provided, the additional sensors may be worn by the person 102 during the subsequent stage.

Notably also, sleep apnea is diagnosed at a low rate. Polysomnography (PSG) is a relatively expensive diagnostic, with large operating expenses and involves a long manual annotation process which is both manually inefficient and computationally inefficient. From the perspective of a patient, PSG is time consuming and disruptive. Further, 80-90% of patients with clinically relevant sleep apnea are not diagnosed. Most current solutions do not capture inter-night variability in AHI scores. There may be large inter-night variability in AHI scores for some patients. Because the duration of PSG is generally only a single night, it may fail to capture nights where the patient exhibits sleep apnea. Further, most home tests collect data for a limited number of nights.

Further, there is a large patient population overlap between sleep apnea and heart disease. For instance, 49% of patients with atrial fibrillation also have obstructive sleep apnea. For patients with obstructive sleep apnea, the risk of developing arrhythmias increases by as much as double or quadruple. Further, for patients with obstructive sleep apnea, the risk of heart failure is increased by two and a half times. Many conventional home sleep apnea tests have a high failure rate, e.g., greater than 10% are unanalyzable, and thus have limited utility to address such issues. Accordingly, conventional solutions lack automation, convenience, and potential insights provided by the described techniques.

### Sleep Apnea Prediction Using Electrocardiograms and Machine Learning

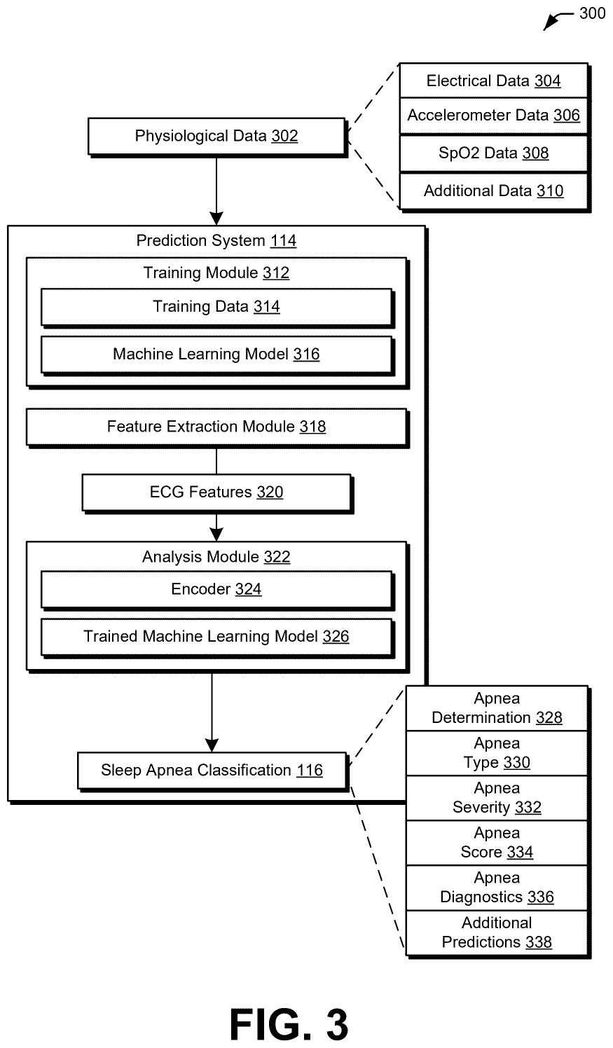

FIG. 3 depicts a non-limiting system in an example implementation 300 of sleep apnea prediction using electrocardiograms and machine learning showing operation of the prediction system 114 of FIG. 1 in more detail.

To begin in this example, the prediction system 114 receives physiological data 302, which may include electrical data 304 (e.g., electrical potential measurements and/or ECG data), accelerometer data 306, SpO2 data 308 (e.g., oxygen saturation data), and/or various additional data 310. In various examples, the physiological data 302 is collected by one or more devices and/or sensors, such as the wearable monitoring device 104. The physiological data 302 can include time-sequenced instances of data, such as continuous data, data collected at predetermined intervals (e.g., per half second interval, per minute interval, per five minute interval, etc.) for the length of an observation period, e.g., a single night, every sleep period during a week, for a month, and so forth. Generally, the physiological data 302 is processed by the prediction system 114 to generate one or more sleep apnea classifications 116 in accordance with the techniques described in more detail below.

For instance, the prediction system 114 includes a training module 312 that is operable to train a machine learning model 316 using training data 314 to perform a sleep apnea classification task. The sleep apnea classification task, for instance, involves generation of a sleep apnea classification 116, such as prediction of whether a user has sleep apnea based on patterns in ECG data. This task may include determination of the type of sleep apnea (e.g., obstructive or central), assessing its severity using metrics like the apnea-hypopnea index (AHI), generating a sleep apnea score, and so forth. Accordingly, the machine learning model 316 is trained to correlate patterns in the physiological data 302, such as various electrical potential measurements, to one or more sleep apnea classifications 116.

The training data 314 may include historical electrical potential measurements, such as ECG data, from a population of users along with corresponding historical outcome data, such as sleep apnea classifications or other outcomes. This data may be collected from clinical studies, sleep labs, or other sources where ECG data and sleep apnea diagnoses are recorded simultaneously. In some cases, the training data 314 may also include additional physiological measurements such as accelerometer data, oxygen saturation levels, and/or additional relevant biomarkers. The training data 314 may be labeled with various sleep apnea-related information, such as the presence or absence of sleep apnea, the type of sleep apnea (e.g., obstructive or central), severity levels, adverse effects caused by sleep apnea, specific AHI scores, and so forth.

In some implementations, different training schemes and/or model architectures are employed based on what the sleep apnea classification 116 is to include. For instance, a composition and structure of the training data 314 may vary depending on the specific type of sleep apnea classification 116 to be generated. In an example in which the sleep apnea classification 116 is to indicate a binary classification of sleep apnea presence (e.g., whether a user has sleep apnea or does not have sleep apnea), the training data 314 may be labeled with yes/no indicators. In an additional or alternative example in which the sleep apnea classification 116 is to include granular predictions such as AHI scores, apnea types, and/or apnea severity levels, the training data 314 includes detailed annotations that pertain to the granular predictions.

In some examples, the training data 314 is be structured to support multi-task learning, where the machine learning model 316 can simultaneously predict multiple aspects of sleep apnea, such as type of sleep apnea and severity of sleep apnea. In additional or alternative examples, the training module 312 trains the machine learning model 316 on a per task basis, such as to implement a first round of training to train the machine learning model 316 to perform a first sleep apnea classification task and a second round of training to train the machine learning model 316 to perform a second sleep apnea classification task. In this way, the techniques described herein support targeted training of the machine learning model 316 for particular tasks, which improves model performance and efficiency to perform discrete aspects of sleep apnea classification.

In one or more implementations, the training module 312 trains the machine learning model 316 using an iterative process of adjusting weights and learning parameters to minimize a loss function. For example, the training module 312 may use backpropagation and/or gradient descent algorithms to update parameters of the model based on a difference between predicted and actual sleep apnea classifications in the training data 314. A learning rate, batch size, and/or number of epochs may be tuned to optimize the performance of the machine learning model 316.

In some examples, techniques such as dropout or regularization may be employed by the training module 312, such as to prevent overfitting. The training process may continue until the model achieves a desired level of accuracy on a validation dataset and/or until a predetermined number of iterations have been completed. This approach allows the machine learning model 316 to learn complex patterns in the physiological data 302 that are indicative of sleep apnea and identify subtle features to develop insights that are not possible using conventional analysis methods. This is by way of example and not limitation, and a variety of suitable training techniques are considered.

Further, as described in more detail above, a variety of architectures/types of the machine learning model 316 are considered. In some aspects, the machine learning model 316 may include a neural network, such as a convolutional neural network (CNN), recurrent neural network (RNN), or a combination thereof. In some cases, the machine learning model 316 incorporates one or more U-Net and/or ResNet architectures, features, or components. The model may also be implemented as an ensemble of different algorithms that combines one or more decision trees, random forests, and/or gradient boosting machines with neural network approaches.

An architecture of the machine learning model 316 is also modifiable based on a desired sleep apnea classification 116. For instance, the machine learning model 316 is configured with one or more attention heads (e.g., classification heads) based on what the sleep apnea classification 116 is to include. By way of example, the machine learning model 316 is trained to distinguish between obstructive sleep apnea (OSA) and central sleep apnea (CSA) and accordingly the machine learning model 316 is configured with two separate classification heads, e.g., one for each apnea type. In an example in which the sleep apnea classification 116 denotes an apnea severity, the machine learning model 316 can be configured with a classification head for each severity designation, e.g., a normal classification head, mild classification head, moderate classification head, and a severe classification head.

Multiple attention heads, for instance, allow the machine learning model 316 to allocate resources to focus on different aspects of the input data to make distinct classifications. Continuing with the above example in which the sleep apnea classification 116 indicates a sleep apnea type, each classification head is trained to detect a specific type of apnea, which enables the prediction system 114 to provide classification specific analysis of the physiological data 302. In this way, the techniques described herein support adaptability of the prediction system 114 to efficiently provide focused diagnostic information.

Once the machine learning model 316 is trained, the physiological data 302 is processed by a feature extraction module 318, which generates ECG features 320. For instance, the feature extraction module 318 preprocesses the physiological data 302 to generate usable (e.g., processable) inputs for the trained machine learning model 326. The feature extraction module 318 may generate the ECG features 320 based on various properties of the ECG signal, such as QRS complex characteristics (e.g., changes in angle of a QRS complex between adjacent measurement intervals), heart rate variability metrics, and/or morphological changes in the ECG waveform. These extracted features can include time-domain, frequency-domain, and non-linear measures that capture relevant information about cardiac activity and respiratory activity.

The feature extraction module 318 is further operable to perform ECG-derived respiration (EDR) to generate the ECG features 320. In some implementations, EDR techniques may be used to extract respiratory information from the ECG signal without dedicated respiratory sensors. The feature extraction module 318 may analyze variations in ECG morphology, such as changes in R-wave amplitude or QRS complex characteristics, which can be correlated to and/or influenced by respiratory activity. These respiratory related properties of the ECG signal may be used to derive respiratory rate, depth, and/or patterns. The EDR-derived features may also be combined with other ECG features 320 to provide a comprehensive set of inputs for the trained machine learning model 326. In this way, the prediction system 114 is able to capture both cardiac and respiratory information from a single ECG signal, which improves detection of correlation between cardiac and respiratory events and is not possible using conventional modalities.

The feature extraction module 318 may further implement a variety of additional techniques such as wavelet decomposition, principal component analysis, and/or other signal processing methods to isolate and quantify relevant aspects of the ECG signal. For example, an R-R interval series may be analyzed to derive heart rate variability parameters, while QRS amplitude and area measurements can provide information about respiratory-induced changes in the ECG. The feature extraction module 318 may further optimize and/or refine the ECG features 320, such based on a discriminative ability of the ECG features 320 to detect particular sleep apnea events.

Once extracted, an analysis module 322 configures the ECG features 320 for input to a trained machine learning model 326 (e.g., the machine learning model 316 once output by the training module 312) using an encoder 324. The encoder 324 is configurable to process and compress input data into a compact representation and can include one or more of a convolutional encoder, recurrent encoder, transformer encoder, one or more autoencoder variants, and so forth. The encoder 324, for instance, generates compressed representations from the ECG features 320 that can be efficiently processed by the trained machine learning model 326. In an example, the encoder 324 reduces a dimensionality of the ECG features 320 while preserving relevant information, creating a compact representation that serves as a suitable input to the trained machine learning model 326.

The analysis module 322 then generates a sleep apnea classification 116 for output by processing the encoded ECG features 320 using the trained machine learning model 326. The sleep apnea classification 116 may include a variety of information such as an apnea determination 328 that indicates presence of sleep apnea, an apnea type 330 that indicates different types of sleep apnea, apnea severity 332 indicating a degree of sleep apnea, an apnea score 334 providing a numerical quantification of sleep apnea, apnea diagnostics 336 offering diagnostic information such as predicted adverse effects due to apnea, and/or additional predictions 338 that provide supplementary analysis results.

In one or more examples, the apnea determination 328 further includes a confidence interval, e.g., a confidence in the sleep apnea classification 116. The apnea score 334 may include an apnea-hypopnea index (AHI) score that quantifies a number of apnea events and hypopnea events per hour of sleep of the person 102 during the observation period. The apnea type 330 may distinguish between types of sleep apnea including but not limited to obstructive sleep apnea (OSA), central sleep apnea (CSA), or a combination of OSA and CSA.

The apnea severity 332, for instance, indicates whether a user associated with the physiological data 302 has no sleep apnea, mild sleep apnea, moderate sleep apnea, severe sleep apnea, etc. The apnea diagnostics 336 can indicate whether a user is at risk for developing sleep apnea and/or whether the use is predicted to experience adverse effects associated with sleep apnea, e.g., daytime fatigue, snoring, low blood oxygen, atrial fibrillation (AFib), or other arrhythmias, to name just a few.

Additionally or alternatively, the sleep apnea classification 116 includes an indication of a correlation between a sleep apnea event and an additional physiological event, such as a cardiac event, respiratory event, neurological event, and so forth. For instance, the trained machine learning model 326 is operable to identify and analyze relationships between apnea occurrences and various cardiac, respiratory, or neurological phenomena. For example, the sleep apnea classification 116 may indicate that apnea events are more likely to occur during periods of increased heart rate variability for a particular user, and/or may indicate a temporal association between apnea events and specific cardiac arrhythmias, such as episodes of atrial fibrillation. Such insights offer a comprehensive view of physiological responses to and causes of apnea events.

In various examples, the physiological data 302 further includes one or more of the accelerometer data 306 or SpO2 data 308. These additional measurements may be input to the machine learning model 316 along with the electrical data 304 to predict the sleep apnea classification 116. Accordingly, the techniques described herein support multi-modality predictions that provide insights not capable using conventional techniques.

For example, the analysis module 322 leverages accelerometer data 306 to enhance accuracy of the sleep apnea classification 116. For instance, the prediction system 114 may process the accelerometer data 306 to identify periods of physical inactivity or reduced movement to generate a sequence of sleep for the user. The prediction system 114 further segments the electrical data 304 into sleep and wake periods based on the sequence of sleep and filters out data related to wake periods. In this way, the trained machine learning model 326 focuses analysis on relevant time frames when sleep apnea events are likely to occur which results in generation of accurate sleep apnea classifications 116 and conservation of computational resources.

Additionally, the accelerometer data 306 may be used to detect body position changes during sleep, which can influence occurrence and/or severity of sleep apnea events. By incorporating such positional information, the trained machine learning model 326 may generate accurate sleep apnea classifications 116 that take into account a relationship between body position and apnea events. For instance, the trained machine learning model 326 is able to process the accelerometer data 306 to generate a sleep apnea classification 116 that indicates a correlation between user behaviors, e.g., a body position while sleeping, and apnea events.

In some implementations, the SpO2 data 308 may be utilized to enhance the accuracy of and/or validate the sleep apnea classification 116. For instance, the analysis module 322 may process the SpO2 data 308 in conjunction with the electrical data 304 to identify potential apnea events. The trained machine learning model 326 may be configured to detect sudden drops in oxygen saturation levels, which may coincide with apnea episodes, and correlate the drops with changes in the ECG signal. By combining these data sources, the prediction system 114 is operable to distinguish between different types of sleep-disordered breathing events with enhanced accuracy.

By way of example, obstructive sleep apnea events may be characterized by continued respiratory effort as indicated by the ECG-derived respiration signal coupled with a drop in SpO2, while central sleep apnea events may show a lack of respiratory effort and a decrease in oxygen saturation. This multi-modal approach enables nuanced and accurate sleep apnea classifications 116, which reduces incidence of false positives and provides additional context for severity and type of apnea events detected.

In at least one example, the sleep apnea classification 116 may include an indication of an efficacy of sleep apnea treatment, such as the effectiveness of a continuous positive airway pressure (CPAP) machine. To support this functionality, the prediction system 114 may be configured to analyze physiological data 302 collected before and after initiation of CPAP therapy for a user. The trained machine learning model 326 may process pre-treatment and post-treatment electrical data 304, accelerometer data 306, and/or SpO2 data 308 to assess changes in sleep apnea patterns and/or severity.

For instance, the analysis module 322 may leverage the trained machine learning model 326 to compare a frequency and/or duration of apnea events, oxygen saturation levels, and/or sleep quality metrics before and after use of the treatment. The sleep apnea classification 116 may then include a treatment efficacy score, indicating a degree of improvement in sleep apnea symptoms. This score may be based on factors such as reduction in AHI, increased oxygen saturation, improved sleep continuity, and so forth. The trained machine learning model 326 may also generate predictions about long-term treatment outcomes and suggest adjustments to CPAP settings based on the analyzed data. Accordingly the techniques described herein further enable assessment of treatment efficacy to support personalized recommendations to improve patient outcomes.

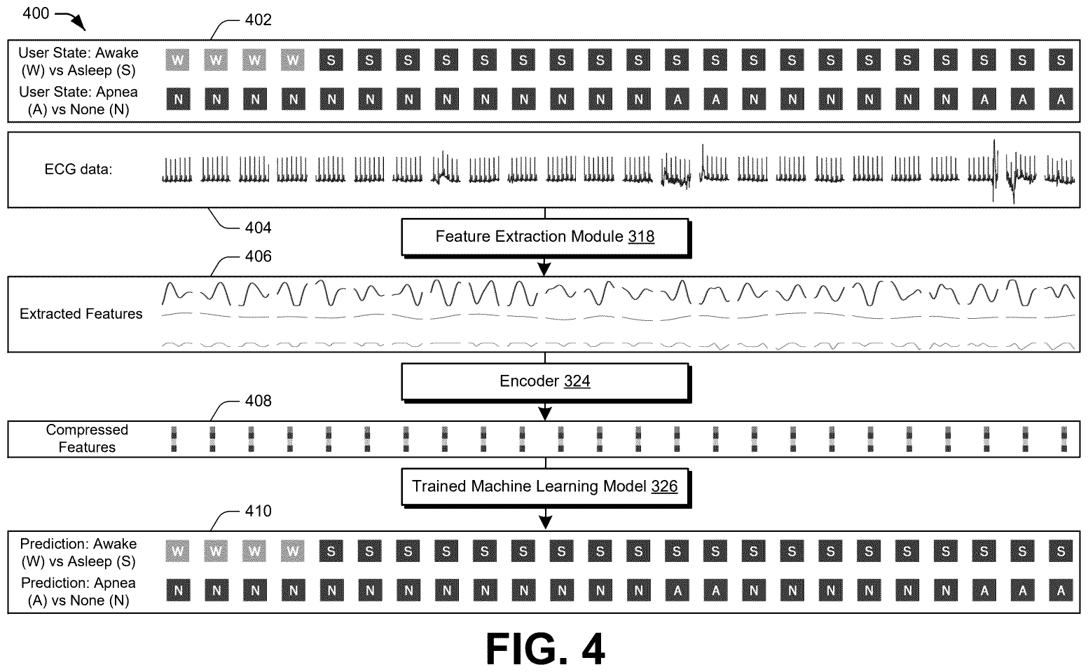

FIG. 4 depicts a non-limiting example 400 of sleep apnea prediction using electrocardiograms and machine learning in which electric potential measurements are processed by a trained machine learning model to generate predictions.

In the illustrated example 400, a user condition 402 is depicted that represents an actual state of a user as either awake or asleep, and whether apnea or no apnea is present, such as during a portion of an observation period. As depicted “W” indicates the user is awake, while “S” indicates a period of sleep. “N” represents no apnea present, while “A” represents an apnea event. The user condition 402, for instance, is representative of a “ground truth” state of the user during the observation period.

Further illustrated is an ECG sequence 404 showing ECG waveform data collected from the user by the wearable monitoring device 104 during the observation period. In accordance with the techniques described herein, the feature extraction module 318 processes the ECG sequence 404 to generate an extracted feature sequence 406. The extracted feature sequence 406 contains derived various features from the ECG waveform, e.g., ECG features 320. As illustrated, a variety of ECG features 320 are extracted. In at least one example, the feature extraction module 318 is configured to perform EDR as part of generation of the extracted feature sequence 406.

The extracted feature sequence 406 is processed by the encoder 324, which compresses the features into a compressed feature sequence 408, represented as a series of discrete markers in this example. The compressed feature sequence 408 is then provided as input to the trained machine learning model 326. The trained machine learning model 326 analyzes the compressed feature sequence 408 and generates an output that includes a prediction sequence 410. In this example, the trained machine learning model 326 includes a 1D U-Net trained for a sleep apnea classification task, e.g., to correlate patterns in electrical potential measurements to sleep apnea classifications. In an additional or alternative example, the trained machine learning model 326 includes a RESNET-based encoder and one or more transformer-based classification heads for sleep/wake and/or apnea event detection.

The prediction sequence 410 in this example 400 includes two rows of predictions. A top row indicates whether the user is predicted to be awake or asleep, and a bottom row indicates predicted apnea events versus no apnea. As illustrated, the predictions in the prediction sequence 410 correspond temporally to the user condition 402 and accurately match the ground truth user state. In this example, the prediction sequence 410 represents model predictions for a single interval, e.g., a thirty-minute interval. As described in the following example, the prediction system 114 is operable to seamlessly concatenate multiple prediction sequences for multiple intervals to provides a holistic representation of an observation period that includes multiple intervals.

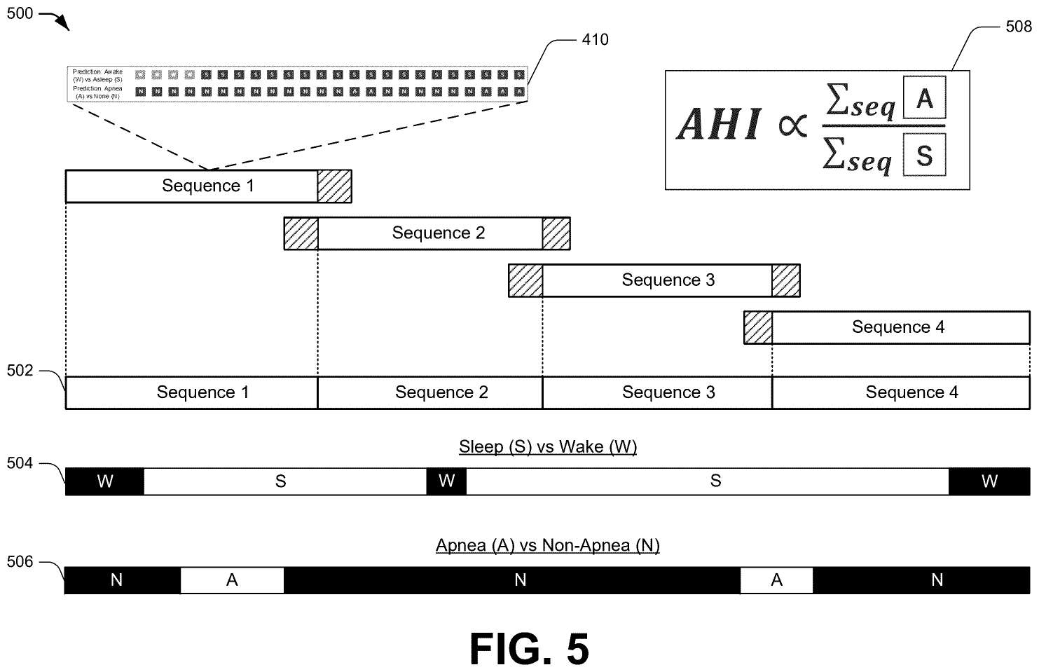

FIG. 5 depicts a non-limiting example 500 of sleep apnea prediction using electrocardiograms and machine learning in which sequences of intermediate outputs of the machine learning model are concatenated to generate a model output.

In this example, multiple prediction sequences are generated, such as the prediction sequence 410 generated in FIG. 4. The prediction system 114 processes multiple overlapping prediction sequences to form a continuous representation of the observation period. For example, the prediction system 114 leverages the trained machine learning model 326 to generate and concatenate a first sequence (e.g., the prediction sequence 410), a second sequence, a third sequence, and a fourth sequence into a concatenated sequence 502. In one or more examples, this includes detection and splicing of overlapping portions of temporally adjacent sequences. Accordingly, the concatenated sequence 502 represents an extended period of time that includes multiple thirty-minute intervals, e.g., two hours. This process is repeatable for a duration of the observation period.

The prediction system 114 further analyzes the concatenated sequence 502 to derive a sleep behavior sequence 504. The sleep behavior sequence 504 distinguishes between wake periods (W) and sleep periods(S) across the observation period. The prediction system 114 further processes the concatenated sequence 502 to generate an apnea behavior sequence 506. The apnea behavior sequence 506 identifies apnea events (A) versus non-apnea periods (N) throughout the observation period.

Based on the sleep behavior sequence 504 and the apnea behavior sequence 506, the prediction system 114 leverages the trained machine learning model 326 to generate a sleep apnea classification 116 that includes an apnea score 334. The apnea score 334, for instance, includes an AHI score computed in accordance with the illustrated AHI formula 508 to quantify a number of apnea events and hypopnea events per hour of sleep of the person 102 during the observation period. In this way, the prediction system 114 enables continuous monitoring and analysis while maintaining temporal consistency during data processing. This approach allows the system to analyze data across multiple nights of monitoring, such as up to 14-30 days in some implementations.

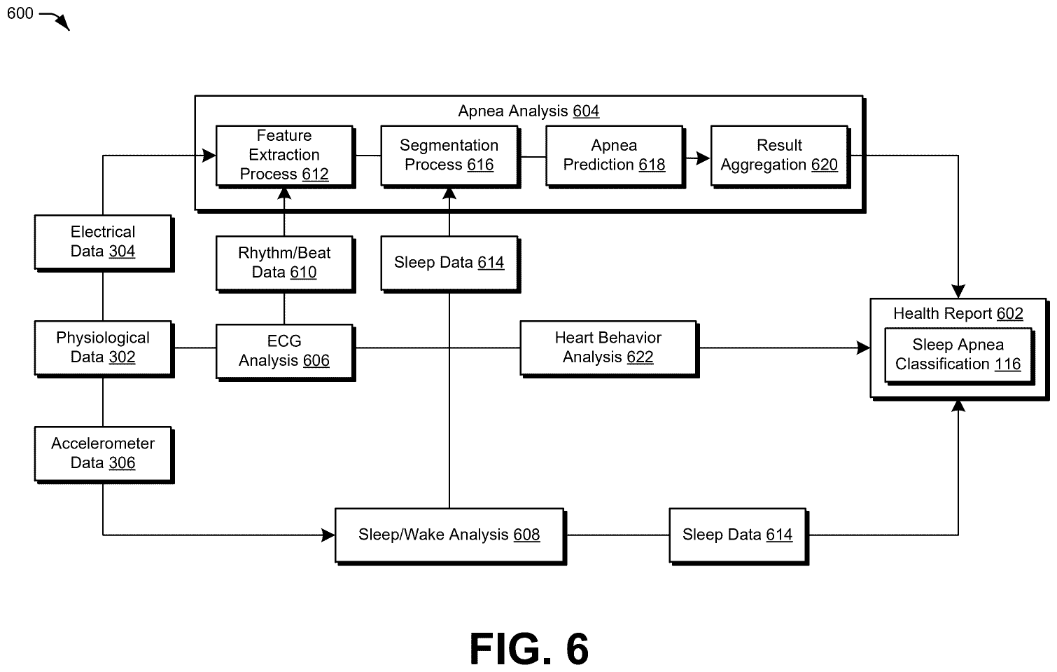

FIG. 6 depicts a system 600 in a non-limiting example of sleep apnea prediction using electrocardiograms and machine learning in which the system generates a comprehensive health report for output that includes the sleep apnea classification.

To begin in this example, the system 600 receives physiological data 302 as input, which includes electrical data 304 and accelerometer data 306. In various embodiments, the physiological data 302 includes additional measurements, such as SpO2 data 308. The physiological data 302 is subject to several analysis processes within the system 600 to generate a health report 602 such as an apnea analysis 604, an ECG analysis 606, and a sleep/wake analysis 608. Various components and hardware as described herein are operable to implement/facilitate the analysis processes.

For instance, the electrical data 304 is first processed at an ECG analysis 606 component to generate rhythm/beat data 610. The rhythm/beat data 610 may include various information about the electrical data 304, such as but not limited to heart rate variability, R-R intervals, QRS complex characteristics (e.g., QRS peak angle), and/or other temporal and/or morphological features extracted from the electrical data 304. The rhythm/beat data 610 is passed to a feature extraction process 612 of the apnea analysis 604 subsystem as well as to a heart behavior analysis 622 component for additional processing.

The accelerometer data 306 is analyzed by a sleep/wake analysis 608 component to generate sleep data 614. The sleep data 614, for instance, indicates a sleep sequence for a user during an observation period, such as when the user is likely awake or likely asleep. The sleep data 614 is used by the apnea analysis 604 subsystem and contributes to the final sleep apnea classification 116, and in various examples is directly represented in the health report 602.

The apnea analysis 604 subsystem processes the electrical data 304, the rhythm beat data 610, and the sleep data 614 in one or more stages. For instance, a feature extraction process 612 extracts relevant features (e.g., ECG features 320) based on the electrical data 304 and/or the rhythm beat data 610. In one or more examples, the apnea analysis 604 subsystem leverages the feature extraction module 318 to perform this functionality.

The extracted features then undergo a segmentation process 616, such as to divide a continuous ECG signal and/or other derived data into distinct time periods or segments for analysis. In an example, this includes partitioning data collected during the observation period into sleep and wake periods, such as based on the sleep data 614. In some examples, the segmentation process 616 is performed by a first pass of the trained machine learning model 326 to generate the segmented data.

An apnea prediction 618 component then analyzes the segmented data to identify potential apnea events. In some implementations, the apnea prediction 618 may be performed by a second pass of the trained machine learning model 326, such as to generate one or more sleep apnea classifications 116. The results from the apnea prediction 618 processes are combined in a result aggregation 620 stage. The result aggregation 620, for instance, synthesizes different analyses and/or various sleep apnea classifications 116 generated by the trained machine learning model 326 to generate a comprehensive sleep apnea classification that incorporates information from multiple data sources and/or processing steps.

In parallel with the apnea analysis 604, a heart behavior analysis 622 component processes outputs from the ECG analysis 606. This analysis may detect cardiac features such as one or more arrhythmias during the observation period based on the physiological data 302. The system 600 combines outputs from the heart behavior analysis 622, the result aggregation 620 from the apnea analysis 604, and the sleep data 614 from the sleep/wake analysis 608 to generate the health report 602. The health report 602, for instance, visual depicts aspects of the sleep apnea classification 116 and/or additional physiological predictions.

Further, because the system 600 processes various types of physiological data 302, the health report 602 and/or the sleep apnea classification 116 can include a variety of multimodal information, such as a variety of insights and/or correspondences between apnea data, heart behavior data, and/or sleep data 614. For instance, the health report 602 can include one or more insights such as correlations between apnea events and cardiac arrhythmias, estimates of potential future health effects based on detected apnea patterns, assessments of apnea severity and type (e.g., obstructive vs. central), and analyses of sleep quality and breathing patterns over time.

The health report 602 may also provide visualizations overlaying apnea events with heart rate data to show potential relationships between the predictions. Additionally, the report can include recommendations for follow-up testing and/or suggested interventions based on the detected apnea characteristics. By combining multiple data sources and analysis techniques, the system 600 provides a comprehensive assessment of sleep apnea and related cardiac events, enabling healthcare providers to make informed decisions about patient care and potential interventions.

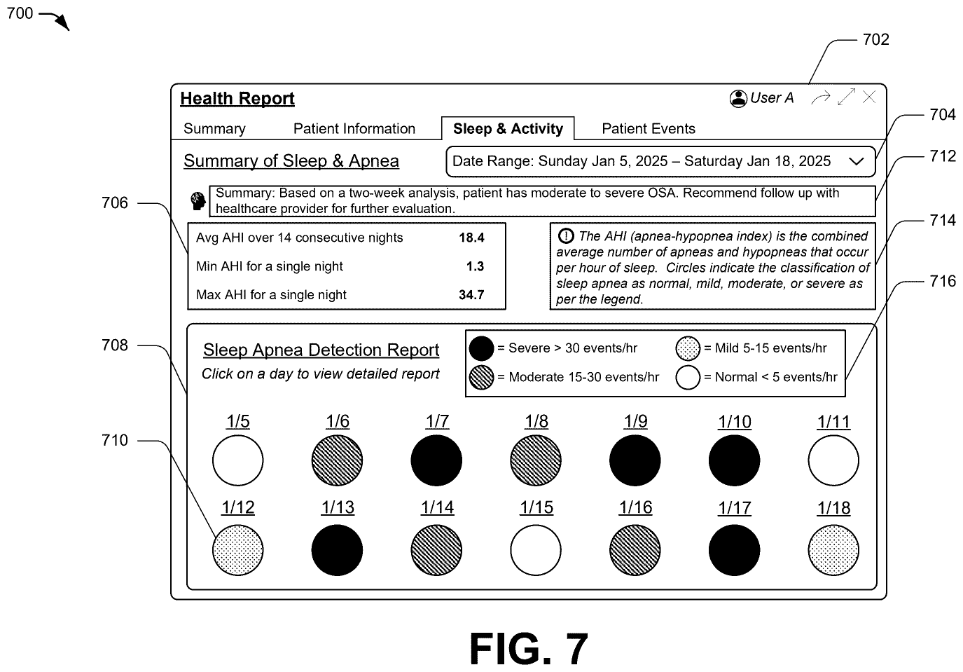

FIG. 7 depicts a non-limiting example 700 of sleep apnea prediction using electrocardiograms and machine learning in which a report that includes the sleep apnea classification is output in a user interface.

As depicted in the illustrated example 700, a user interface 702 depicts a sleep and activity tab of a health report that includes various insights derived from one or more sleep apnea classifications 116 generated in accordance with the techniques described herein. For instance, the health report represents results from an observation period in which a user wears a wearable monitoring device 104 to capture various physiological data 302. In some examples, the health report is generated during the observation period. Additionally or alternatively, the health report is generated after the observation period has concluded.

In this example, the interface 702 includes a date selector 704, a summary indicator 706, a detection report 708 that includes various daily indicators 710, an analysis summary 712, an information panel 714, and an indicator legend 716 included in the detection report 708. The date selector 704, for instance, allows selection of a date range for viewing the analysis. The date selector 704 may enable a user to choose specific dates or predefined time periods, such as a week or a month, to display corresponding sleep apnea data.

The summary indicator 706 provides statistical data that includes average, minimum, and maximum AHI values across the selected observation period. The AHI values, for instance, are determined in accordance with the techniques described herein, such as via processing of electrical potential data by the trained machine learning model 326. The summary indicator 706 may offer a quick overview of sleep apnea severity trends over time. The information panel 714 provides explanatory content about the AHI measurements and classification system and may help users understand a meaning of the presented data and how it relates to overall sleep health.

The interface 702 further includes a detection report 708 that displays daily indicators 710 showing sleep apnea severity levels for each day over a two-week period. In some cases, the daily indicators 710 may use color coding or other visual cues to represent different severity levels, such as normal, mild, moderate, or severe sleep apnea. For instance, the indicator legend 716 defines symbols and/or colors used to represent different severity levels of sleep apnea events.

For example, the indicator legend 716 indicates color coding used to denote normal, mild, moderate, or severe sleep apnea occurrence for each daily indicator 710. The severity levels, for instance, are determined in accordance with the techniques described herein, such as via processing of electrical potential data by the trained machine learning model 326. This visual representation allows users to easily track changes in sleep apnea patterns over time.

An analysis summary 712 presents an overview of analysis results and recommendations. The analysis summary 712 may include interpretations of the sleep apnea classification data and suggestions for follow-up actions based on the detected patterns. In some examples, the analysis summary 712 is generated by the trained machine learning model 326 based on processing of previously generated sleep apnea classifications 116. For instance, the trained machine learning model 326 received as input sleep apnea classifications 116 that indicate daily sleep apnea severity and analyzes these to generate the analysis summary 712.

In the illustrated example the analysis summary 712 indicates that the patient “likely has moderate to severe OCA” and that a further evaluation is recommended. Thus, the interface 702 includes a variety of information and insights generated by the prediction system 114 using the techniques described herein. By presenting the sleep apnea classification in a visual and interactive format, the interface 702 enables users and healthcare providers to easily interpret complex sleep data and make informed decisions about sleep health and potential interventions.

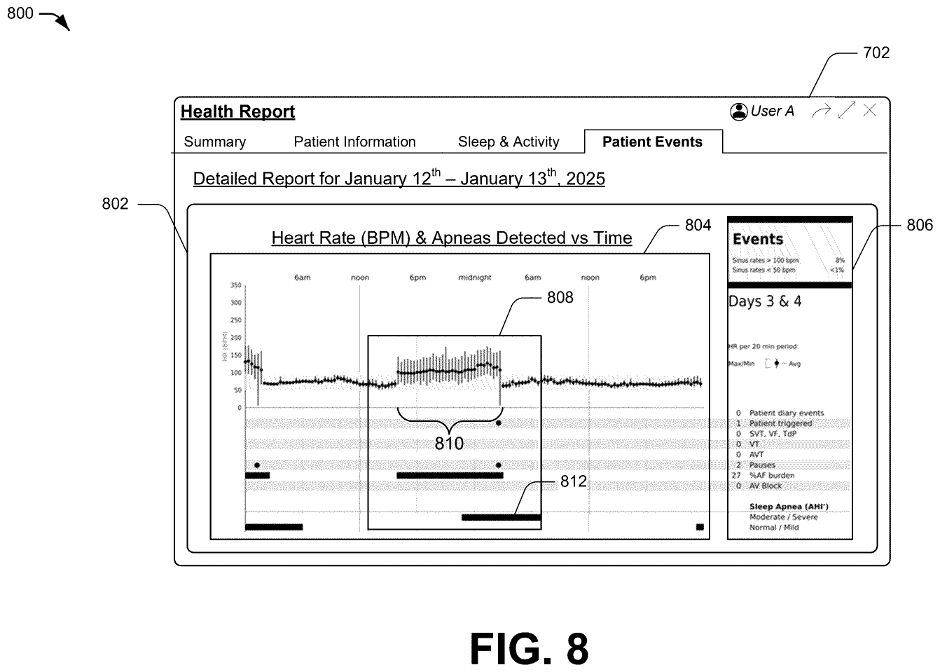

FIG. 8 depicts a non-limiting example 800 of sleep apnea prediction using electrocardiograms and machine learning in which a report that includes the sleep apnea classification is output in a user interface. The example 800, for instance, represents a continuation of the example 700 discussed above with respect to FIG. 7. For example, an input is received to select a particular daily indicator 710, which causes the prediction system 114 to display a daily report section 802, such as in a “Patient Events” tab.

The daily report section 802 presents data for a specific time period, e.g., a single night of sleep. In this example, the daily report section 802 displays detailed information from January 12th to January 13th. The daily report section 802 may allow users to view sleep apnea data and related physiological measurements for individual days or for portions of the observation period.

The daily report section 802 includes a graph 804 and an information panel 806. The graph 804, for instance, is titled “Heart Rate (BPM) & Apneas Detected vs Time” and depicts heart rate variations and apnea events over a 24-hour period. This graph 804 provides a visual representation of correlations between heart rate patterns and occurrences of apnea events and supports visualization of information associated with individual apnea events.

For instance, a graph excerpt 808 highlights a particular time period where notable variations in cardiac and apnea events occurred and includes a visual indication of a correlation between a sleep apnea classification 116 and a cardiac arrythmia. As illustrated, the graph excerpt 808 depicts an atrial fibrillation event 810 that corresponds to a sleep apnea event 812 detected by the prediction system 114. Accordingly, by leveraging electrical potential data to detect both apnea and cardiac events, the prediction system 114 provides enhanced insights into the complex interplay between respiratory and cardiovascular systems during sleep. Such insights are not possible using conventional techniques that are reliant on separate data types.

In some examples, this further conserves computational resources that would otherwise be expended processing multiple input data types from multiple independent sensor modalities. Accordingly, by generating multi-modality insights (as well as correlations between such insights) based on a single data type the techniques described herein are able to improve operations of devices that implement the prediction system 114.

The information panel 806 further displays various event types and corresponding statistics for the displayed time interval. For instance, the information panel 806 provides a summary of a variety of data points and events to complement the visual information presented in the graph 804. These may include patient events, cardiac events/statistic, respiratory events, suspected sleep apnea severity classifications, and so forth. For instance, the information panel 806 indicates that the sleep apnea event 812 is designated as a “Moderate/Severe” apnea event.

Accordingly, the daily report section 802, graph 804, and information panel 806 of the interface 702 present a multifaceted view of the sleep apnea data as well as other physiological observations, e.g., cardiac data. This comprehensive presentation may facilitate interpretation of complex sleep and cardiac data, potentially leading to earlier detection of sleep apnea and support effective management of related health issues.

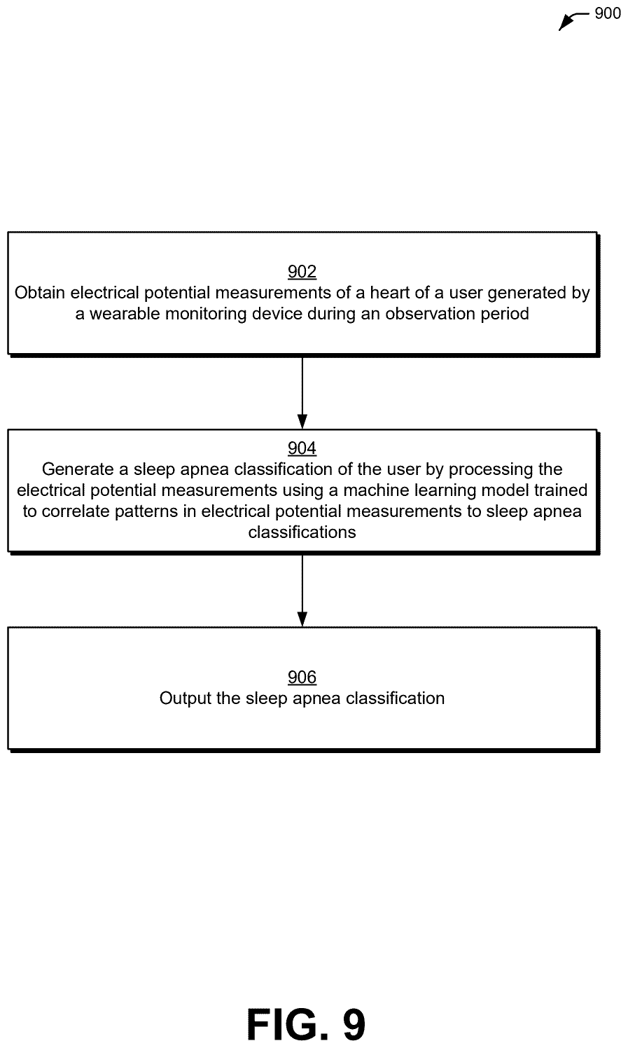

FIG. 9 depicts a flow diagram depicting an algorithm as a step-by-step procedure 900 in an example implementation that is performable by a processing device to generate a sleep apnea classification based on electrical potential measurements.

To begin in this example, electrical potential measurements of a heart of a user are obtained (block 902). The electrical potential measurements, for instance, are produced by the wearable monitoring device 104 during an observation period. In various examples, the wearable monitoring device 104 detects electrical activity of the heart of the person 102 using one or more of the sensors 202 and produces the electrical potential measurements 110 based on the detected activity.

A sleep apnea classification of the user is generated by processing the electrical potential measurements using a machine learning model (block 904). The machine learning model 316, for instance, is trained using historical electrical potential measurements and historical outcome data of a user population to perform a sleep apnea classification task. The sleep apnea classification task can include correlation of patterns in electrical potential measurements to sleep apnea classifications. For example, the trained machine learning model 326 receives the electrical potential measurements 110 as input and predicts the sleep apnea classification 116.

The sleep apnea classification is then output (block 906). For example, the prediction system 114 causes display of the sleep apnea classification 116 via a user interface. Alternatively or additionally, the sleep apnea classification 116 may be incorporated into one or more reports, e.g., a health report 602. In various examples, notifications or alerts related to the sleep apnea classification 116 are output via a computing device associated with a person 102 or a healthcare provider of the person 102.

The previous examples describe multiple instances of machine-learning models such as the machine learning model 316 and/or the trained machine learning model 326. Machine-learning models refer to a computer representation that is tunable (e.g., through training and retraining) based on inputs without being actively programmed by a user to approximate unknown functions, automatically and without user intervention. In particular, the term machine-learning model includes a model that utilizes algorithms to learn from, and make predictions on, known data by analyzing training data to learn and relearn to generate outputs that reflect patterns and attributes of the training data. Examples of machine-learning models include neural networks, convolutional neural networks (CNNs), long short-term memory (LSTM) neural networks, generative adversarial networks (GANs), decision trees, support vector machines, linear regression, logistic regression, Bayesian networks, random forest learning, dimensionality reduction algorithms, boosting algorithms, deep learning neural networks, etc.

A machine-learning model, for instance, is configurable using a plurality of layers having, respectively, a plurality of nodes. The plurality of layers are configurable to include an input layer, an output layer, and one or more hidden layers. Calculations are performed by the nodes within the layers via hidden states through a system of weighted connections that are “learned” during training of the machine-learning model to implement a variety of tasks.

In order to train the machine-learning model, training data is received that provides examples of “what is to be learned” by the machine-learning model, i.e., as a basis to learn patterns from the data. The machine-learning system, for instance, collects and preprocesses the training data that includes input features and corresponding target labels, i.e., of what is exhibited by the input features. The machine-learning system then initializes parameters of the machine-learning model, which are used by the machine-learning model as internal variables to represent and process information during training and represent interferences gained through training. In an implementation, the training data is separated into batches to improve processing and optimization efficiency of the parameters of the machine-learning model during training.

The training data is then received as an input by the machine-learning model and used as a basis for generating predictions based on a current state of parameters of layers and corresponding nodes of the model, a result of which is output as output data, e.g., a search result, prompt, and so forth.

Training of the machine-learning model can include calculating a loss function to quantify a loss associated with operations performed by nodes of the machine learning model. The calculating of the loss function, for instance, includes comparing a difference between predictions specified in the output data with target labels specified by the training data. The loss function is configurable in a variety of ways, examples of which include regret, Quadratic loss function as part of a least squares technique, and so forth. Configuration of the training data is usable to support a variety of usage scenarios and model tasks, such as one or more sleep apnea classification tasks. A variety of other examples are also contemplated, including the U-Net and/or ResNet architectures as previously described.

The various functional units illustrated in the figures and/or described herein are implemented in any of a variety of different manners such as hardware circuitry, software or firmware executing on a programmable processor, or any combination of two or more of hardware, software, and firmware. The methods provided are implemented in any of a variety of devices, such as a general-purpose computer, a processor, or a processor core. Suitable processors include, by way of example, a general purpose processor, a special purpose processor, a conventional processor, a digital signal processor (DSP), a graphics processing unit (GPU), a parallel accelerated processor, a plurality of microprocessors, one or more microprocessors in association with a DSP core, a controller, a microcontroller, Application Specific Integrated Circuits (ASICs), Field Programmable Gate Arrays (FPGAs) circuits, any other type of integrated circuit (IC), and/or a state machine.

In one or more implementations, the methods and procedures provided herein are implemented in a computer program, software, or firmware incorporated in a non-transitory computer-readable storage medium for execution by a general-purpose computer or a processor. Examples of non-transitory computer-readable storage mediums include a read only memory (ROM), a random-access memory (RAM), a register, cache memory, semiconductor memory devices, magnetic media such as internal hard disks and removable disks, magneto-optical media, and optical media such as CD-ROM disks, and digital versatile disks (DVDs).

It should be understood that many variations are possible based on the disclosure herein. Although features and elements are described above in particular combinations, each feature or element is usable alone without the other features and elements or in various combinations with or without other features and elements.

What is claimed is:

1. A method implemented by a processing device comprising:
obtaining electrical potential measurements of a heart of a user generated by a wearable monitoring device during an observation period;
generating a sleep apnea classification of the user by processing the electrical potential measurements using a machine learning model trained to correlate patterns in electrical potential measurements to sleep apnea classifications; and
outputting the sleep apnea classification.

2. The method of claim 1, wherein the sleep apnea classification includes an indication describing a state of the user during the observation period as having sleep apnea or not having sleep apnea.

3. The method of claim 1, wherein the machine learning model is configured to determine a severity of sleep apnea based on the electrical potential measurements, and the sleep apnea classification includes an indication describing a state of the user during the observation period as having no sleep apnea, mild sleep apnea, moderate sleep apnea, or severe sleep apnea.

4. The method of claim 1, wherein the machine learning model is configured to generate a sleep apnea score based on the electrical potential measurements, and the sleep apnea classification includes an apnea-hypopnea index (AHI) score that quantifies a number of apnea events and hypopnea events per hour of sleep of the user during the observation period.

5. The method of claim 1, wherein the machine learning model is configured to determine a type of sleep apnea based on the electrical potential measurements, and the sleep apnea classification includes an indication describing a state of the user during the observation period as having obstructive sleep apnea (OSA) or central sleep apnea (CSA).

6. The method of claim 1, wherein the generating the sleep apnea classification includes extracting one or more electrocardiogram (ECG) features based on the electrical potential measurements and providing the one or more electrocardiogram features to the machine learning model as input.

7. The method of claim 1, further comprising obtaining one or more additional physiological measurements from the wearable monitoring device and wherein the generating the sleep apnea classification includes inputting the one or more additional physiological measurements to the machine learning model as input.

8. The method of claim 7, wherein the one or more additional physiological measurements include accelerometer data or oxygen saturation measurements.

9. The method of claim 1, further comprising training the machine learning model to perform a sleep apnea classification task using historical electrical potential measurements and historical outcome data of a user population as training data.

10. A processing device comprising:
one or more processors; and
memory having stored computer-readable instructions that are executable by the one or more processors to perform operations comprising:
obtaining electrical potential measurements of a heart of a user collected by a wearable monitoring device during an observation period;
generating a sleep apnea classification of the user by providing the electrical potential measurements to a machine learning model as input, the machine learning model trained using historical electrical potential measurements and historical outcome data of a user population to perform a sleep apnea classification task; and
outputting the sleep apnea classification in a user interface of the processing device.

11. The processing device of claim 10, wherein the wearable monitoring device includes one or more sensors to collect accelerometer data and the generating the sleep apnea classification includes processing the accelerometer data by the machine learning model to determine a sequence of sleep of the user during the observation period.

12. The processing device of claim 10, wherein the wearable monitoring device includes one or more sensors to collect oxygen saturation data and the generating the sleep apnea classification includes processing the oxygen saturation data by the machine learning model to validate the electrical potential measurements.

13. The processing device of claim 10, wherein the sleep apnea classification is output during the observation period.

14. The processing device of claim 10, wherein the sleep apnea classification is output following the observation period.

15. The processing device of claim 10, wherein the sleep apnea classification includes an indication of a type and a severity of sleep apnea.

16. The processing device of claim 10, the operations further comprising generating, by the machine learning model, an indication of one or more predicted future adverse effect of sleep apnea based on the electrical potential measurements.

17. A system comprising:
a wearable monitoring device that is wearable by a user to detect one or more physiological measurements of the user during an observation period, the one or more physiological measurements including electrical potential measurements of a heart of the user; and
a computing device configured to:
receive the one or more physiological measurements from the wearable monitoring device;
generate a sleep apnea classification of the user by processing the one or more physiological measurements by a machine learning model trained to correlate patterns in electrical potential measurements to sleep apnea classifications; and
output the sleep apnea classification.

18. The system of claim 17, wherein the physiological measurements further include accelerometer data collected during the observation period or oxygen saturation data collected during the observation period.

19. The system of claim 17, wherein the sleep apnea classification includes details associated with an individual apnea event detected during the observation period.

20. The system of claim 17, the computing device further configured to:
detect one or more cardiac arrythmias during the observation period based on the one or more physiological measurements;
generate, using the machine learning model, a correlation between the sleep apnea classification and the one or more cardiac arrythmias; and
generate a visual indication for output by the computing device of the correlation.
